SEQUENCE LISTING (20 sequences)

SEQ ID NO: 1 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSNTNYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSNTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTNYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 344 aa, Artificial Sequence
  Variant human immunoglobulin single variable domain-Fc fusion polypeptide
PEVQLQVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKRLEWVSEISPSGSYTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTTVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKRLEWVSEISPSGSNTNYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 344 aa, Artificial Sequence
  Variant human immunoglobulin single variable domain-Fc fusion polypeptide
PEVQLLVSGGGLVQPGGSLRLSCAASGFTFKAYPMMWVRQARGQRLEWVSEISPSGSYTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 344 aa, Artificial Sequence
  Variant human immunoglobulin single variable domain-Fc fusion polypeptide
PEVQLLVSGGGLVQPGGSLRLSCAASGFTFKAYPMMWVRQARGQRLEWVSEISPSGSNTNYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTYYADSVKGRFTISRDNSKNTLSLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 99 aa, Homo Sapiens
PEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 13 - protein, 99 aa, Homo Sapiens
PQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 14 - protein, 227 aa, Homo Sapiens
PTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKR

SEQ ID NO: 15 - protein, 347 aa, Artificial Sequence
  Variant human immunoglobulin single variable domain-Fc fusion polypeptide
PEVQLLVSGGGYESLVQPGGSLRLSCAASGFTFKAYPMMWVRQAPGKGLEWVSEISPSGSKTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTKYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 344 aa, Artificial Sequence
  Variant human immunoglobulin single variable domain-Fc fusion polypeptide
PEVQLLVSGGGLVQPGGSLRLSCAASGFTFKAYPMMWVRQARGQRLEWVSEISPSGSNTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTYYADSVKGRFTISRDNSKNTLKLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYKYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 344 aa, Artificial Sequence
  Variant human immunoglobulin single variable domain-Fc fusion polypeptide
PEVQLLVSGGGLVQPGGSLRLSCAASGFTFKAYPMMWVRQAPGKGLEWVSEISPSGSYTYNADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2013/0310281 A1
(43) Pub. Date: Nov. 21, 2013

(54) NOVEL ANTIGEN BINDING PROTEINS

(71) Applicants: Emma R. Harding, Stevenage (GB); Ekaterini Kotsopoulou, Stevenage (GB); Alan Peter Lewis, Stevenage (GB); Susannah Thornhill, Stevenage (GB); Mark Uden, Stevenage (GB)

(72) Inventors: Emma R. Harding, Stevenage (GB); Ekaterini Kotsopoulou, Stevenage (GB); Alan Peter Lewis, Stevenage (GB); Susannah Thornhill, Stevenage (GB); Mark Uden, Stevenage (GB)

(73) Assignee: Glaxo Group Limited, Brentford, Middlesex (GB)

(21) Appl. No.: 13/981,972

(22) PCT Filed: Jan. 27, 2012
(86) PCT No.: PCT/EP2012/051374

§ 371 Date: Jul. 26, 2013

Related U.S. Application Data
(60) Provisional application No. 61/438,742, filed on Feb. 2, 2011.

(51) Int. Cl. C07K 16/22

(52) U.S. Cl. CPC C07K 16/22
USPC 506/18; 530/389.2; 530/387.3

(57) ABSTRACT

The present invention provides novel antigen-binding proteins derived from human germline V_{H} domains, having improved expression and improved biophysical characteristics.

This invention relates to novel antigen binding proteins with increased expression titres and/or improved biophysical characteristics. More particularly, the invention relates to immunoglobulin (antibody) single variable domains, in particular isolated V_{H} domains (domain antibodies/dAbs) and fusion proteins comprising such V_{H} domains, with improved expression and reduced propensity to aggregate. Such antigen binding proteins may be pharmaceutically active and may be useful in the treatment or prophylaxis of disease. The invention also relates to methods for improving the biophysical characteristics of such antigen binding proteins.

### BACKGROUND OF THE INVENTION

Domain antibodies are the smallest known antigen-binding fragments of antibodies comprising the robust variable regions of the heavy or light chains of immunoglobulins (V_{H} and V_{L}, respectively) (reviewed, for example, in Holt et al. (2003) Trends in Biotechnology Vol.21, No.11 p. 484-490).

A number of domain antibodies, including human antibody light and heavy chain variable domain antibodies (V_{L} and V_{H} dAbs), camelid V_{H}H domains (nanobodies) and shark new antigen receptors (V-NAR), that bind to specific target molecules/antigens are being developed as immunotherapeutics (see, for example, Enever et al. Current Opinion in Biotechnology (2009); 20: 1-7). Development of a domain antibody as an immunotherapeutic follows the same approach that has been established in the case of single chain Fvs and involves screening a dAb phage display library to select for target binding polypeptides, followed by affinity maturation to improve antibody affinity (K_{D}). Suitable methods are described, for example in WO 2005/118642.

Domain antibodies can exist and bind to target in monomeric or multimeric (especially dimeric) forms, and can be used in combination with other molecules for formatting and targeting approaches. Such targeting approaches include building antigen-binding constructs optionally having multiple domains for engaging several targets at the same time. For example, an antigen-binding construct having multiple domains can be made in which one of the domains binds to serum proteins such as albumin. Domain antibodies that bind serum albumin (AlbudAbs™) are described, for example, in WO05/118642 and can provide the domain fusion partner an extended serum half-life in its own right. A monomer dAb may be preferred for certain targets or indications where it is advantageous to prevent target cross-linking (for example, where the target is a cell surface receptor such as a receptor tyrosine kinase e.g. TNFR1). In some instances, binding as a dimer or multimer could cause receptor cross-linking of receptors on the cell surface, thus increasing the likelihood of receptor agonism and detrimental receptor signalling. For certain targeting approaches involving a multidomain antigen-binding construct, it may be preferable to use a monomer dAb e.g. when a dual targeting molecule is to be generated, such as a dAb-AlbudAb™ where the AlbudAb binds serum albumin, as described above, since dimerizing dAbs may lead to the formation of high molecular weight protein aggregates, for example.

dAbs may also be conjugated to other molecules, for instance in the form of a dAb-Fc fusion protein (for example, WO2008/149150), or as an antibody-dAb fusion protein (for example, WO2009/068649). Conjugated dAbs such as a dAb-Fc fusion may be useful if effector functions, e,g. ADCC or CDC are preferred.

Whereas camelid and shark single variable domains are likely to be immunogenic in humans, fully human V_{H} or V_{L} dAbs are less likely to raise an immune response and have great potential as therapeutic proteins. However, human V_{H} dAbs can exhibit characteristics which are not optimal for expression and manufacture. It is believed that some of these characteristics may result from the exposure of hydrophobic residues which would ordinarily interface with the V_{L} chain, potentially leading to reduced solubility and thermodynamic stability. Autonomous single variable domains from the Camelid family (V_{HH}), in contrast, are highly soluble, which was initially attributed to four highly conserved mutations in the interface (Muyldermans et al (Protein Eng., 1994:7 1129-35). It is also thought that an extended CDRH3 loop in VhH domains may interact with this hydrophobic interface region (Desmyter et al. Nat. Struct. Biol. (1996) 3:803-811).

Efforts have been made to improve the biophysical characteristics of human V_{H} dAbs by the process of “camelisation”, with some success, although this process has the potential to increase immunogenicity of the molecule, and can have further untoward effects. For instance, Davies and Reichmann (FEBS Lett., 1994:339 285-290) report the camelisation of human V_{H} domains, and noted that the incorporation of three hydrophilic residues in place of hydrophobic interface residues (G44E/L45R/W47G) resulted in increased solubility, but a significant decrease in expression.

Barthelemy et al (JBC, 2008:6 3639-3654 and also WO2007/134050) described an analysis of the light chain interface of human V_{H} domains, and concluded that CDRH3 in human V_{H} domains did not interact with the light chain interface, and thus CDR3 diversity was not constrained by structural demands. The authors propose various substitutions of the interface residues to improve hydrophilicity. Jespers et al (J. Mol. Biol. 2004:337, 893-903, observed an increase in hydrophilicity of a human V_{H} dAb (HEL4) with the introduction of a glycine residue at position 35, in CDR1, and concluded that a cavity formed by this residue was able to accommodate the hydrophobic Trp47 side chain. Reiter et al. (J. Mol. Biol. 1999:290, 685-698) describe the creation of a stabilized VH library based on the randomization of residues in CDR3. The library was based on a natural framework scaffold of a mouse monoclonal antibody which comprised a lysine residue at position 44.

It would be desirable to improve the biophysical characteristics of immunoglobulin single variable domains, to improve stability and expression, and to reduce aggregation.

### SUMMARY OF THE INVENTION

The present invention describes variant immunoglobulin single heavy chain variable domain amino acid sequences (V_{H}) in which substitutions to the amino acid sequence are made which stabilize the monomeric state of the immunoglobulin single variable domain. These variant V_{H} domains offer significant advantages in downstream processing and formulation. More surprisingly still, these substitutions can also result in a significant increase in expression titres. The or each substitution involves the replacement of certain positions with residues having an increased hydrophilicity, and/or reduced aggregation propensity. In particular embodiments the or each substitution is made within the second CDR or hypervariable region (according to Kabat). Typically the substituted residue or residues replace residues in the V_{H} domain derived from a first human germline sequence with residues which are naturally occurring in other human germline sequences. Thus, the risk of an immunogenic response upon administration to humans may be diminished.

The present invention therefore has application in the improvement of the biophysical characteristics and expression of V_{H} domain antibodies. The invention also has application in the design of libraries of V_{H} domain and monoclonal antibodies with improved expression and biophysical characteristics, and provides a way to isolate an increased number of candidate dAbs with desirable properties.

The V_{H} domains of the invention are derived from human germline sequences, such as the human

DP-47 germline or the DP-2 germline. Other human germline sequences may be used. Particularly, the V_{H} domains are derived from a human germline V_{H}, in which one or more residues, or one or more framework regions, has been replaced with the corresponding residue from another human germline V_{H}. In particular, the present invention describes a number of mutations that stabilize the monomeric state of DP-47 framework V_{H} domain antibodies.

The substitution(s) made to the immunoglobulin single variable (V_{H}) domains of the invention may improve the expression of the V_{H} domain in a biological (cell-based) expression system. The modification may improve the biophysical characteristics of the V_{H} domain. For example, the modification may increase the aqueous solubility of the V_{H} domain, and/or may reduce the propensity of the V_{H} domain to aggregate.

Accordingly, in a first aspect, the invention provides a variant of a parent polypeptide comprising a V_{H} domain having a human germline framework, the variant comprising a V_{H} domain which differs from the V_{H} domain of the parent polypeptide by a substitution of at least one of amino acid positions 56, 57, 58, 59 and 79, wherein the amino acid at said at least one amino acid position of the variant polypeptide is more hydrophilic or has a reduced aggregation propensity than the substituted amino acid of the parent polypeptide.

Suitably, the one or more residues are replaced with residues having a higher hydrophilicity/lower hydrophobicity. Methods for predicting hydrophobicity are described in Biswas et al. (2003), Eisenberg (1984), Janin (1979), the hydropathy index of Kyte and Doolittle (1982), Rose et al. (1985), Rose and Wolfenden (1993), Wimley and White (1996) and Wolfenden et al. (1981). Hydropathy of amino acids is shown according to the Kyte Doolittle index, in which a higher (more positive) number indicates an increasing hydrophobicity. For the purposes of the present invention, hydrophilicity is assessed according to the Kyte/Doolittle hydropathy index (see FIG. 15).

Suitably, the one or more residues are replaced with residues having reduced aggregation propensity. Algorithms for predicting aggregation on a residue-by-residue basis are described in Chennamsetty et al. (2009), Conchillo-Sole et al. (2007), Fernandez-Escamilla et al (2004), Maurer-Stroh et al. (2010), Mendoza et al. (2010), Pawar et al (2005) and Trovato et al (2007). For the purposes of the present invention, aggregation propensity is assessed according to Pawar et al, at pH 7 (see FIG. 16).

In an embodiment, the or each substituted amino acid residue of the parent polypeptide is replaced with a residue having a higher hydrophilicity. In an embodiment, the or each substituted amino acid residue of the parent polypeptide is replaced with a residue having a reduced aggregation propensity. In an embodiment, the or each substituted amino acid residue is replaced with a residue having a higher hydrophilicity and a lower aggregation propensity.

The locations of CDRs and framework (FR) regions within immunoglobulin molecules, and the numbering system applicable to the residues therein, have been defined by Kabat et al. (Kabat, E. A. et al., Sequences of Proteins of Immunological Interest, Fifth Edition, U.S. Department of Health and Human Services, U.S. Government Printing Office (1991)). In all aspects or embodiments of the invention where amino acid numbering is indicated, positions are assigned in accordance with Kabat.

The variant may comprise a V_{H} domain, optionally as part of a larger polypeptide. Suitably, the variant has a framework region encoded by a human germline antibody gene segment, and comprises a substitution of at least one of amino acid positions 56, 57, 58, 59 and 79.

In an embodiment, the variant has improved biophysical properties, including, increased expression when compared to the parent polypeptide, or increased stability when compared to the parent polypeptide, or increase solubility when compared to the parent polypeptide. In an embodiment, the variant has increased expression and increased stability when compared to the parent polypeptide.

In a particular embodiment, the variant polypeptide has an expression titre which is greater than that of the parent polypeptide under defined (i.e. comparable or identical) conditions by at least 10%, 15%, 20%, 25%, 30%, 40%, 50%, 60%, 70%, 80%, 90%, 100%, 200%, 300%, 400% or 500%.

In an embodiment, the variant is substantially monomeric in solution, or has an increased stability in the monomeric state in comparison to the parent polypeptide. In an embodiment, under defined (i.e. comparable or identical) conditions (e.g. buffer, temperature, pH), the proportion of monomeric variant polypeptide in solution is at least 5%, 10%, 15%, 20%, 25% or 30% greater than the proportion of monomeric parent polypeptide in solution. In a particular embodiment, the variant polypeptide has a propensity to aggregate which is at least 5%, 10%, 15%, 20%, 25% or 30% less than that of the parent polypeptide under defined (i.e. comparable or identical) conditions (e.g. pH and temperature).

In an embodiment, the variant polypeptide comprises a substitution at each of amino acid positions 56, 57, 58, 59 and 79. In other embodiments, a substitution may be made at any combination or permutation of such amino acid positions.

In an embodiment, the variant polypeptide comprises a substitution of at least one, and optionally all, of amino acid positions 56, 57, 58 and 59.

In specific embodiments, the variant polypeptide comprises a substitution at each of positions 56, 58 and 59; 56, 57 and 58; 57, 58 and 59; 56 and 57; 56 and 58; 56 and 59; 57 and 58; 57 and 59; 58 and 59; 56; 57; 58; or 59. In accordance with an embodiment of the invention, each of these substitutions may optionally be combined with a substitution at position 79. In a particular embodiment, there is a single substitution, at amino acid position 56. In another particular embodiment, the single substitution is at amino acid position 58.

In particular embodiments, the substituted residue is replaced with an asparagine or a lysine residue, particularly an asparagine residue.

In an embodiment, the human germline framework is selected from the VH3 subgroup. In a particular embodiment, the human germline framework is the DP-47 germline framework (SEQ ID NO:12).

In one embodiment, the amino acid at said at least one amino acid position of the variant polypeptide is an amino acid residue which exists natively in the equivalent position of an alternative human V_{H} germline framework. In a particular embodiment, the alternative human V_{H} germline framework is the DP-2 germline sequence (SEQ ID NO:13).

In an particular embodiment, the variant polypeptide comprises a V_{H} domain having the framework regions of the human DP-47 germline framework, wherein the amino acid of at least one of positions 56 and 58 has been substituted for a more hydrophilic amino acid, and/or an amino acid with a reduced aggregation propensity. In an embodiment, one, or both of residues 56 and 58 are substituted for a residue selected from the group consisting of Ala, Thr, His, Gly, Ser, Gln, Arg, Lys, Asn, Glu, Pro and Asn, more particularly, Lys, Asn, Glu and Asp, and more particularly still, Asn or Lys.

In an embodiment, the more hydrophilic acid is selected from amino acids having a hydrophobicity of from −0.7 or less, −1.0 or less, −1.5 or less, −2.0 or less, −2.5 or less, −3.5 or less, −4.0 or less or −4.5 or less on the Kyte/Doolittle scale. Particular hydrophilic amino acids useful in the present invention are asparagine and lysine.

In an embodiment, the residue(s) having reduced aggregation propensity are those having an aggregation propensity at pH 7, according to Pawar et al (ibid) of −3.0 or less, −4.0 or less, −4.5 or less, −5.0 or less, −5.5 or less, −6.5 or less, −7.0 or less, −7.5 or less, −8.0 or less, −8.5 or less, −9.0 or less, −9.5 or less, −10.0 or less, −10.5 or less, −11.0 or less, −11.5 or less. Particular amino acids with limited aggregation propensity are Ala, Gly, His, Ser, Gln, Asn, Asp, Lys, Glu, Arg and Pro. Particular amino acids useful in the present invention are serine, asparagine and lysine.

In a specific embodiment, the amino acid at position 56 has been substituted for a more hydrophilic amino acid, optionally Ala, Thr, His, Gly, Ser, Gln, Arg, Lys, Asn, Glu, Pro and Asn, more particularly, Lys, Asn, Glu and Asp, and more particularly still, Asn.

In a specific embodiment, the amino acid at position 58 has been substituted for a more hydrophilic amino acid, optionally Ala, Thr, His, Gly, Ser, Gln, Arg, Lys, Asn, Glu, Pro and Asn, more particularly, Lys, Asn, Glu and Asp, and more particularly still, Asn.

Thus, in one embodiment, one or both of residues 56 and 58 are replaced with Asn (N), and in a particular embodiment, residue 56 is substituted for an Asn (N) residue.

In an embodiment, the variant polypeptide comprises the amino acid sequence of amino acids 1-116 of SEQ ID NO:3 or SEQ ID NO:4, or the full length of SEQ ID NO:3 or SEQ ID NO:4.

In another aspect, the invention provides a V_{H} domain comprising an amino acid sequence of amino acids 1-116 of SEQ ID NO:1, optionally the full length of SEQ ID NO:1, in which at least one of residues at positions 56, 57, 58 and 59 of the V_{H} domain is substituted for a more hydrophilic residue, or for a residue with a reduced propensity to aggregate.

In an embodiment the substitution is of positions 56 and/or 58.

In an embodiment, the V_{H} domain comprises the amino acid sequence of amino acids 1-116 of SEQ ID NO:3 or SEQ ID NO:4.

In another aspect, the invention provides a polypeptide comprising or having the amino acid sequence of amino acids 1-116 of SEQ ID NO:3 or SEQ ID NO:4.

In another aspect, the invention provides a polypeptide comprising or having the amino acid sequence of SEQ ID NO:3 or SEQ ID NO:4.

In another aspect, the invention provides a V_{H} domain comprising framework regions having 85%, 90%, 95%, 96%, 97%, 98%, 99% or 100% identity to a framework encoded by human germline sequence DP-47 (SEQ ID NO:12), wherein position 56 of the V_{H} domain is an asparagine residue, or a lysine residue.

In another aspect, the invention provides a V_{H} domain comprising framework regions having 85%, 90%, 95%, 96%, 97%, 98%, 99% or 100% identity to a framework encoded by human germline sequence DP-47 (SEQ ID NO:12), wherein position 58 of the V_{H} domain is an asparagine residue, or a lysine residue.

In another aspect, the invention provides a V_{H} domain comprising framework regions having 85%, 90%, 95%, 96%, 97%, 98%, 99% or 100% identity to a framework encoded by human germline sequence DP-47 (SEQ ID NO:12), wherein positions 56 and 58 of the V_{H} domain are asparagine residues.

In another aspect, the invention provides a V_{H} domain comprising framework regions having 85%, 90%, 95%, 96%, 97%, 98%, 99% or 100% identity to a framework encoded by human germline sequence DP-47 (SEQ ID NO:12), wherein position 57 of the V_{H} domain is an asparagine residue, or a lysine residue.

In another aspect, the invention provides a V_{H} domain comprising framework regions having 85%, 90%, 95%, 96%, 97%, 98%, 99% or 100% identity to a framework encoded by human germline sequence DP-47 (SEQ ID NO:12), wherein position 59 of the V_{H} domain is an asparagine residue, or a lysine residue.

In further embodiments of the invention described herein, the substitutions hereinbefore described may be combined with further substitutions at positions 41, 43 and 44.

Accordingly, in certain specific embodiments, the V_{H} domain of the invention may comprise an amino acid sequence of amino acids 1-116 of any of SEQ ID NOs: 2, 3, 4, 8, 10 and 11. In a more specific embodiment, the V_{H} domain comprises an amino acid sequence of amino acids 1-116 of any of SEQ ID NOs: 2, 3 or 4. In a particular embodiment, the V_{H} domain comprises an amino acid sequence of amino acids 1-116 of SEQ ID NO:3. In another particular embodiment, the V_{H} domain comprises an amino acid sequence of amino acids 1-116 of SEQ ID NO:4.

In an embodiment, the V_{H} domain has an amino acid sequence of amino acids 1-116 of any one of SEQ ID NOs: 2, 3, 4, 8, 10 or 11, and may further comprise a domain of an antibody constant region. In an embodiment, the V_{H} domain comprises an amino acid sequence of amino acids 1-116 of any of

SEQ ID NOs: 2, 3 or 4, and further comprises a domain of an antibody constant region. In a particular embodiment, the polypeptide has an amino acid sequence as set out in SEQ ID NO:3, or SEQ ID NO:4.

In another aspect, the invention provides an antigen-binding construct comprising a protein scaffold which is linked to an antigen-binding V_{H} domain according to the invention. The construct may comprise additional antigen binding sites for different antigens, such as additional epitope binding domains. In one embodiment the antigen binding construct has specificity for more than one antigen, for example two antigens, or for three antigens, or for four antigens.

The protein scaffold may be an Ig scaffold such as IgG, or IgA scaffold. The IgG scaffold may comprise some or all the domains of an antibody (i.e. C_{H1}, C_{H2}, C_{H3}, V_{H}, V_{L}). The antigen-binding construct of the present invention may comprise an IgG scaffold selected from IgG1, IgG2, IgG3, IgG4 or IgG4PE. In one embodiment, the scaffold is IgG1.

In one embodiment, the scaffold consists of, or comprises, the Fc region of an antibody, or is a part thereof. In one aspect a part of an Fc domain comprises a part of an Fc region having any effector function as described herein, for example an Fc receptor binding activity.

In another aspect, the invention provides a polypeptide comprising a V_{H} domain as hereinbefore described, linked to a domain of a human antibody constant region. In a particular embodiment, the V_{H} domain is conjugated to an Fc domain. Domain antibodies in this format are described in WO2008/149450.

The domain of the antibody constant region may be an antibody Fc region. As used herein the term “Fc” has been used to mean an Fc sequence from an IgG1 (such as the Fc region of SEQ ID NO:14) wherein the sequence starts “THTCPPC” and ends “KR”. Other Fc variants are known in the art and are included within the scope of the present application. Such variants may have a sequence at least 70%, 75%, 80%, 85%, 90%, 95%, 96%, 97%, 98%, 99% or 100% identical to the amino acid Fc sequence of SEQ ID NO:14.

Also provided is a polypeptide comprising an anti-VEGF immunoglobulin single variable domain, linked to an antibody Fc domain, wherein said polypeptide has the amino acid sequence of SEQ ID NO:3.

Also provided is a polypeptide comprising an anti-VEGF immunoglobulin single variable domain, linked to an antibody Fc domain, wherein said polypeptide has the amino acid sequence of SEQ ID NO:4.

Also provided is a polypeptide comprising an anti-VEGF immunoglobulin single variable domain, linked to an antibody Fc domain and which has an amino acid sequence that 70%, 75%, 80%, 85%, 90%, 95%, 96%, 97%, 98%, 99% or 100% identical to the amino acid sequence of SEQ ID NO:1, on the proviso that the anti-VEGF immunoglobulin single variable domain comprises an asparagine at position 56.

A further aspect provides a library comprising immunoglobulin heavy chain variable domain regions in accordance with the invention wherein at least one of amino acid positions 56, 57, 58, 59 and 79 has been substituted. A library comprising V_{H} domains capable of binding to a target antigen, wherein at least one of amino acid positions 56, 57, 58, 59 and 79 is not diversified, and wherein said at least one amino acid position is selected from residues having a hydrophilicity of less than -1.0 on the Kyte/Doolittle scale, or residues having an aggregation propensity of less -2.12 at pH 7 (according to Pawar et al.).

A further aspect which may be mentioned provides a library comprising immunoglobulin heavy chain variable domain regions wherein at least one of amino acid positions 56, 57, 58, 59 and 79 is not diversified.

In an embodiment, at least one of amino acid positions 56 and 58 is not diversified. In this embodiment, positions 57, 59 and 79 may be diversified.

In an embodiment, position 56 is not diversified in the library. In this embodiment, position 56 may be an asparagine or lysine residue. In another embodiment, position 58 is not diversified in the library. In this embodiment, position 58 may be an asparagine or lysine residue.

In another embodiment, both positions 56 and 58 are not diversified, and are selected, independently, from asparagine and lysine residues.

In another embodiment, position 56, 57, 58 and 59 are not diversified, wherein position 56 is selected from asparagine, lysine or tyrosine, position 57 is tyrosine, position 58 is selected from asparagine, lysine or tyrosine, and position 59 is selected from asparagine, lysine or tyrosine. In a particular embodiment, positions 56, 58 or 59 are selected from asparagine and lysine.

In one embodiment, the library is a V_{H} DP47 library.

Another aspect provides a library for expressing variant heavy chain variable domain regions in accordance with the invention comprising a list of nucleic acid sequences encoding said heavy chain variable domains.

There is also provided a library of nucleic acids encoding a polypeptide or a immunoglobulin heavy chain single variable domain in accordance with the invention.

In another aspect, the invention provides a list or a library in accordance with the invention wherein said library further comprises diversity in the CDR regions. Diversity in CDR regions can be generated by suitable methods.

In another aspect, the invention provides a method of modifying a polypeptide comprising an immunoglobulin single heavy chain variable (V_{H}) domain, the method comprising substituting at least one amino acid at positions 56, 58 and 59 of the V_{H} domain with an amino acid which is more hydrophilic than the substituted amino acid.

In an embodiment, the method increases expression titre and/or monomeric stability of the polypeptide.

In another aspect, the invention provides a method of increasing the expression titre of a polypeptide comprising an immunoglobulin single heavy chain variable (V_{H}) domain, the method comprising substituting at least one amino acid at positions 56, 58 and 59 of the V_{H} domain with an amino acid which is more hydrophilic than the substituted amino acid.

In another aspect, the invention provides a method of increasing the monomeric stability of a polypeptide comprising an immunoglobulin single heavy chain variable (V_{H}) domain, the method comprising substituting at least one amino acid at positions 56, 58 and 59 of the V_{H} domain with an amino acid which is more hydrophilic than the substituted amino acid.

In an embodiment of the above-described methods, the method comprises determining the identity of the amino acid residues at one or more of positions 56, 58 and 59, assessing the hydrophilicity of the or each residue, and replacing one or more of said residues with a more hydrophilic residue.

The invention also provides a polynucleotide encoding a V_{H} domain, polypeptide or antigen-binding construct according to the invention. The invention also provides an expression vector comprising such a polynucleotide, and a host cell comprising such an expression vector.

In another aspect, the invention provides a method of producing a V_{H} domain or polypeptide according to the invention, comprising culturing a host cell under conditions conducive to the expression of the V_{H} domain or polypeptide. The method may further comprise purifying the expressed V_{H} domain or polypeptide. In an embodiment, the host cell is a mammalian host cell, such as a CHO cell. In another embodiment, the host cell is a microbial host cell, such as E. Coli.

In a further aspect, the invention provides a method of treating a patient suffering from diseases associated with VEGF signalling, such as cancer and/or ocular diseases such as Diabetic Macular

Edema, Wet AMD, Diabetic retinopathy, RVO or corneal neovascularisation, comprising administering an effective amount of a V_{H} domain, polypeptide or antigen-binding construct as disclosed herein.

### BRIEF DESCRIPTION OF THE DRAWINGS

FIG. 1: Amino acid sequences for the “parental” VEGF dAb-Fc molecule (DOM15-26-593-Fc) and variant molecules generated. The amino acid substitutions are underlined and in bold font. Each of sequences 1 to 11 and 14 comprise a lysine (K) residue at the C terminal thereof. The C-terminal lysine of the Fc region is commonly “clipped” during expression/post-translational modification. Accordingly, it is to be understood that the C-terminal lysine residue may not be present in the mature V_{H} domains and polypeptides according to the invention. In other words, each of sequences 1 to 11 and 14, as referred to herein, may end with the residues SPG, instead of SPGK) as opposed to as shown in the figures, c.f. SEQ ID NO:14).

FIG. 2: Growth curves of bulk-transfected populations for first round of mutations. ChK2 cells (CHO-K1sv cells harbouring an artificial chromosome) were bulk-transfected with plasmids encoding parental DOM15-26-593-Fc or variant molecules together with a plasmid encoding a mutated lambda integrase. Stable transfectants were selected in bulk and seeded into 500 ml unfed production curves. Two batches were seeded at subsequent passages and for each molecule they were seeded in duplicate. The data shows increased dAb-Fc fusion yields following 7 or 8 days culture for the DOM15-26-593-Fc Y56N Y58N variant as compared to parental. The G44R, L5Q and L108T substitutions had no effect on productivity at this stage.

FIG. 3: Overgrowth assay for SCC of first round of mutations. The bulk-transfected parental DOM15-26-593-Fc—and variant cell lines (Y56N and Y58N, G44R, L5Q and L108T) were single-cell cloned. The resulting clones were assessed for productivity following scale-up to 24-well plates using an overgrowth assay to account for differences in cell line growth characteristics. The majority of clones for the DOM15-26-593-Fc Y56N Y58N variant were found to express increased levels of dAb-Fc fusion as compared to the parental molecule and the remainder of the variant forms.

FIG. 4: Growth curves of SCCs of first set of mutations. dAb-Fc productivity during batch and enriched batch growth curves of clonal cell lines expressing the parental DOM15-26-593-Fc molecule and variant forms. The DOM15-26-593-Fc Y56N Y58N variant had a significantly increased expression, with a titre of 2-2.7 fold higher than that of the parental cell line under equivalent conditions. Expression from the remaining variant cell lines was at equivalent or lower levels than achieved for the parental molecule.

FIG. 5A-E: Effect of mutations on biophysical properties. The dAb-Fc variants expressed in CHO cells were captured using an agarose based Protein A chromatography resin, washed with phosphate buffer pH 7 and eluted in 0.1M Sodium acetate pH 3.5. One eluate sample was adjusted directly to pH 7.0 (labelled ‘flash’). The remaining eluate was pH adjusted to 3.0 using hydrochloric acid, followed by titration to 7.0 using sodium hydroxide, taking a sample every 0.5 pH unit for analysis. (A): Percent protein loss on pH adjustment of protein A eluate. (B) Turbidity during rising pH (A600 measurement across pH (3-7). (C) SEC-HPLC data analysis showing % aggregate at a range of pH (3-7). (D) Tm as determined by differential scanning calorimetry (DSC). (E) Binding data (% binding and Affinity by BIACORE®) . Titration data indicates improved solubility of the DOM15-26-593-Fc Y56N Y58N variant at pH 7.0 and a corresponding 7-12% protein loss compared to 28-50% with the parental DOM15-26-593-Fc molecule (FIGS. 1 & 2). Aggregate levels vary significantly between variant and parental at low pH (3-3.5). Tm values when measured by differential scanning calorimetry are indistinguishable between the parental DOM15-26-593-Fc and the variant forms.

FIG. 6A-B: Growth curves of second set of mutants. Bulk transfections and clonal cell lines were generated for the second series of variants in an identical manner to the first series and productivity assessed in enriched batch growth curves (expression from these lines, including parental, was observed to be considerably lower than for the first series of clones, likely a result of decreased transfection efficiency). (A) shows the data for the bulk transfections and (B) for the top clonal lines for each variant. Mutation of either of the tyrosine residues at positions 56 or 58 individually were found to increase dAb-Fc yield, with a two-fold increase in productivity observed for the DOM15-26-593-Fc Y58N mutant as compared to parental molecule. Increased productivity was also observed for the P4IR & K43Q & G44R & Y56N & Y58N variant only for the done and for the P41R & K43Q & G44R variant only for the bulk transfection.

FIG. 7A-C: Effects of second round of mutations on molecule aggregation/precipitation. Capture and elution was performed as for FIG. 5. (A): Turbidity during rising pH (A600 measurement across pH (3-7). (B) Soluble protein loss at a range of pH (3-7). (C) % Aggregate and specific activity.

FIG. 8A-C: SEC-HPLC data for the parental (WT) and Y56N variants. The dAb-Fc variants were dialysed or pH adjusted into a range of buffers commonly used in downstream processing or formulation between pH 3.0 and 7.8. (A) and (B) %aggregate comparison by SEC-HPLC for the Y56N mutant and the WT between pH 3.0 and 4.5 in 25mM sodium citrate and 30mM sodium acetate respectively. (C) %aggregate comparison by SEC-HPLC for Y56N and the WT following pH increase from 6.8 to 7.8. In all conditions tested Y56N displayed an increased % monomer and corresponding decreased % aggregate compared to the WT molecule.

FIG. 9A-B: Percent protein recovery following pH adjustment of the parental (WT) and Y56N variants. The dAb-Fc variants in sodium acetate pH3.0-4.5 were titrated to pH 6.8-7.8 in a variety of buffers. Significant precipitation was observed for many samples. Soluble protein was isolated and percent recoveries calculated. (A) % recovery comparison between the WT and Y56N mutant molecule following pH increase from pH3.0-4.5 to pH6.8 or pH 7.8. (B) % recovery comparison for Y56N and the WT following pH titration to pH 7.5 using either NaOH, Tris, or Tris containing excipients. In all cases the recovery of the Y56N variant was significantly higher than the WT, with Y56N showing good solubilities under the conditions tested.

FIG. 10: Expression data and biophysical properties for the third set of variants. Three sets of bulk transfections were generated for the third set of variant dAb-Fc molecules (see second column for the numbers of transfection sets performed for each molecule). Polyclonal pools were subsequently generated and evaluated in enriched batch growth curves for productivity relative to the parental molecule. Data presented in column three represents the % increase or range thereof relative to parent of the mean titres from each separate set of transfections. The highest-expressing polyclonal pools were then single-cell cloned and productivity of the resulting clones assessed using a 24-well overgrowth assay (as previously described) and in shake flasks in enriched batch growth curves. To assess the biophysical properties of the parent and variant molecules, capture and elution was performed as for FIG. 5 and the percent protein loss on pH adjustment of the protein A eluate to pH 7 is shown. Aggregate levels following column elution are also compared. Mutation of either of the tyrosine residues at positions 56 or 58 to either asparagine (as demonstrated previously) or lysine residues were found to increase dAb-Fc yield and improve molecule solubility. Improved biophysical properties were also observed for the Y59N variant and decreased levels of aggregate were observed for the T57K variant; however clonal lines for both these molecules expressed at lower levels than the parent.

FIG. 11: Soluble protein loss upon titration from pH 3-7 for the third set of variants. Capture and elution as in FIG. 5. Shows percent loss of soluble protein on pH adjustment of Protein A eluate.

FIG. 12A-C: Expression in polyclonal pools for three sets of variant dAb-Fc molecules. Variants were generated for three further dAb-Fc molecules, replacing the residues at either position 56 or 58 with asparagines. Following bulk transfection and scale-up, productivity was assessed for each parental and mutant molecule in polyclonal pools in enriched batch shake flasks. For all molecule tested, mutation of the residue at position 56 to an asparagine was demonstrated to increase productivity. For the 15-8 and 7r29 molecules, mutation of the amino acid at position 58 to an aspargine was also shown to enhance expression levels relative to parent.

FIG. 13A-C: Overgrowth Assay for SCC of three sets of variant dAb-Fc molecules. The bulk-transfected parental and variant cell lines were single-cell cloned. The resulting clones were assessed for productivity in 24-well plates using an overgrowth assay as previously described. (A-C) For all three dAb-Fc molecules the X56N and Y58N variants were found to express at higher levels than the parent lines.

FIG. 14A-C—Soluble protein loss upon titration from pH 3-7 for the three sets of variant dAb-Fc molecules. Capture and elution as in FIG. 5. Shows percent loss of soluble protein on pH adjustment of protein A eluate. (A) 15-8 wild type and two variants (B) 7r-29 wild type and two variant (C) 21-23 wild type and two variants.

FIG. 15: Hydropathy of amino acids. Based on evaluations by Kyte and Doolittle, J Mol Biol. 1982;157:105-132

FIG. 16: Aggregation propensity of amino acids. Based on Pawar, A. P., Dubay, K. F., Zurdo, J., Chiti, F., Vendruscolo, M. and Dobson, C. M. (2005) Prediction of “aggregation-prone” and “aggregation-susceptible” regions in proteins associated with neurodegenerative diseases. J. Mol. Biol. 350:379-392.

### DETAILED DESCRIPTION OF THE INVENTION

Unless defined otherwise, all technical and scientific terms used herein have the same meaning as commonly understood by one of ordinary skill in the art (e.g, in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

As used herein, “immunoglobulin” refers to a family of polypeptides which retain the immunoglobulin fold characteristic of antibody molecules, which contain two β-sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules which possess binding domains. In one embodiment, the present invention relates to antibodies.

As used herein “domain” refers to a folded protein structure which retains its tertiary structure independently of the rest of the protein. Generally, domains are responsible for discrete functional properties of proteins and in many cases may be added, removed or transferred to other proteins without loss of function of the remainder of the protein and/or of the domain. By single antibody variable domain or immunoglobulin single variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

A V_{H} DP-47 germline sequence, also written as “DP-47”, is an immunoglobulin domain derived from the human framework VH3 family. The DP-47 V_{H} is a human germline variable domain also known as

IGHV3-23 or M99660, Likewise, a V_{H} DP-2. germline sequence, also written as “DP-2”, is a human germline variable domain also known as IGHV1.-58 or M29809. Further human germline V_{H} sequences are described in Tomlinson et al, J. Mol. Biol. (1992) 227 776-798, the content of which is incorporated herein in its entirety.

The phrase “immunoglobulin single variable domain” refers to an antibody variable domain (V_{H}, V_{HH}, V_{L}) or binding domain that specifically binds an antigen or epitope independently of different or other V regions or domains. An immunoglobulin single variable domain can be present in a format (e.g, homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{H}H dAbs. Camelid V_{H}H are immunoglobulin single variable domain polypeptides that are derived from species including camel, llama, alpaca, dromedary, and guanaco, which produce heavy chain antibodies naturally devoid of light chains. Camelid V_{H}H may be humanized. Correspondingly, human V_{H} may be camelized.

Antibody heavy chain domains are indicated by VH or V_{H}, VHH, V_{H}H or V_{HH}. A “variant” with reference to an immunoglobulin light chain single variable domain is one which comprises the amino acid sequence of a naturally occurring, germ line or parental immunoglobulin light chain but differs in one or more amino acids. That is a “variant” comprises one or more amino acid differences when compared to a naturally occurring sequence or “parental” sequence from which it is derived.

Suitably a “parental” sequence is a naturally occurring immunoglobulin heavy chain single variable domain sequence, a germ line immunoglobulin heavy chain sequence or an amino acid sequence of an immunoglobulin heavy chain single variable domain which has been identified to bind to an antigen of interest. In one embodiment, the parental sequence may be selected from a library such as a 4G or 6G library described in WO2005093074 and WO04101790, respectively.

A “lineage” refers to a series of immunoglobulin single variable domains that are derived from the same “parental” clone. For example, a lineage comprising a number of variant clones may be generated from a parental or starting immunoglobulin single variable domain by diversification, site directed mutagenesis, generation of error prone or doped libraries. Suitably binding molecules are generated in a process of affinity maturation. Suitable assays and screening methods for identifying an immunoglobulin light chain single variable domain are described, for example in WO2010/094723 and WO2010/094722, for example. A “parental” sequence includes immunoglobulin single variable domains such as DOM15-26-593 (dAb), and DOM15-26-593-Fc (dAb-Fc), which are described in WO2008/149147 and WO2008/149150, respectively. These molecules are also described herein, as SEQ ID NO:1 (wherein amino acids 1-116 represent DOM15-26-593, and the full sequence represents DOM15-26-593-Fc. Suitably, said variants may also include variation in the CDR sequences, such variation contributing to differences in antigen specificity.

In one embodiment, the parental sequence may be modified in accordance with the invention so as to improve one or more of the biophysical properties or characteristics, such as solution state (measured, for example by SEC and/or SEC MALLS or AUC), solubility and thermostability (measured, for example, by DSC). In one embodiment, the variant has an amino acid substitution at one or more amino acid positions within the immunoglobulin heavy chain single variable domain, and is substantially monomeric. By “substantially monomeric” it is meant that the predominant form of the single variable domain is monomeric in solution.

Solution state can be measured by SEC (as described herein), SEC-MALLS, or AUC (analytical ultra-centrifugation). Suitably, the invention provides a (substantially) pure monomer. In one embodiment, the variant polypeptide is at least 70, 75, 80, 85, 90, 95, 98, 99, 99.5% pure or 100% pure monomer. Suitably where monomeric state is measured by SEC, the variant polypeptide concentration may be in the range of 5 to 10 μM.

The solubility of a protein depends upon the nature of the protein surface and its interaction with the surrounding solvent. Exposed residues with hydrophilic side chain may improve solubility due to favourable interactions with the aqueous/hydrophilic buffer. A change in solubility may result from a change in conditions for example by changing pH, buffer conditions, temperature or concentration of the protein.

Solubility of the protein as described herein refers to the amount of protein that is able to be retained in a solution state. Higher concentrations of protein may lead to precipitation in solution, which may be observed visually by the observation of a change from a clear solution to one that is cloudy or contains flakes/powder. This may be assessed by absorbance at 600 nm which measures the amount of light scattered by particles in solution. The insoluble protein may also be removed for example by filtration or centrifugation, and the concentration of the protein remaining measured by absorbance at 280 nm. Comparison of this to the initial concentration in initial conditions allows a percentage protein loss or percentage soluble protein to be calculated.

As used herein an “antibody” refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from, for example, serum, B-cells, hybridomas, transfectomas, yeast or bacteria.

As described herein an “antigen” is a molecule that is bound by a binding domain according to the present invention. Typically, antigens are bound by antibody ligands and are capable of raising an antibody response in vivo. It may be, for example, a polypeptide, protein, nucleic acid or other molecule.

As used herein, the phrase “target” refers to a biological molecule (e.g, peptide, polypeptide, protein, lipid, carbohydrate) to which a polypeptide domain which has a binding site can bind. The target can be, for example, an intracellular target (e.g, an intracellular protein target), a soluble target (e.g, a secreted), or a cell surface target (e.g, a membrane protein, a receptor protein). Suitably a target is a molecule having a role in a disease such that binding said target with a binding molecule in accordance with the invention may play a role in amelioration or treatment of said disease. The target antigen may be, or be part of, polypeptides, proteins or nucleic acids, which may be naturally occurring or synthetic. In this respect, the ligand of the invention may bind the target antigen and act as an antagonist or agonist (e.g., EPO receptor agonist). One skilled in the art will appreciate that the choice is large and varied. They may be for instance, human or animal proteins, cytokines, cytokine receptors, enzymes, co-factors for enzymes or DNA binding proteins.

In an embodiment, the target is Vascular endothelial growth factor (VEGF). VEGF is a secreted, heparin-binding, homodimeric glycoprotein existing in several alternate forms due to alternative splicing of its primary transcript (Leung et al., 1989, Science 246: 1306). VEGF is also known as vascular permeability factor (VPF) due to its ability to induce vascular leakage, a process important in inflammation.

Thus, one aspect of the invention is a method for treating a disease associated with VEGF signalling in a patient comprising the steps of:
a) identifying a patient with a disease associated with VEGF signalling;
b) providing a V_{H} domain, polypeptide or antigen-binding construct of the invention; and
c) administering the V_{H} domain, polypeptide or antigen-binding construct of the invention to the patient;
   whereby the a disease associated with VEGF signalling in the patient is treated.

An important pathophysiological process that facilitates tumour formation, metastasis and recurrence is tumour angiogenesis. This process is mediated by the elaboration of angiogenic factors expressed by the tumour, such as VEGF, which induce the formation of blood vessels that deliver nutrients to the tumour. Accordingly, an approach to treating certain cancers is to inhibit tumour angiogenesis mediated by VEGF, thereby starving the tumour. AVASTIN (bevacizumab; Genetech, Inc.) is a humanized antibody that binds human VEGF that has been approved for treating colorectal cancer. An antibody referred to as antibody 2C3 (ATCC Accession No. PTA 1595) is reported to bind VEGF and inhibit binding of VEGF to epidermal growth factor receptor 2. Targeting VEGF with currently available therapeutics is not effective in all patients, or for all cancers. Thus, a need exists for improved agents for treating cancer and other pathological conditions mediated by VEGF e.g. vascular proliferative diseases (e.g. Age related macular degeneration (AMD)).

VEGF has also been implicated in inflammatory disorders and autoimmune diseases. For example, the identification of VEGF in synovial tissues of RA patients highlighted the potential role of VEGF in the pathology of RA (Fava et al., 1994, J. Exp. Med. 180: 341: 346; Nagashima et al., 1995, J. Rheumatol. 22: 1624-1630). A role for VEGF in the pathology of RA was solidified following studies in which anti-VEGF antibodies were administered in the murine collagen-induced arthritis (CIA) model. In these studies, VEGF expression in the joints increased upon induction of the disease, and the administration of anti-VEGF antisera blocked the development of arthritic disease and ameliorated established disease (Sone et al., 2001, Biochem. Biophys. Res. Comm. 281: 562-568; Lu et al., 2000, J. Immunol. 164: 5922-5927). Hence targeting VEGF may also be of benefit in treating RA, and other conditions e.g. those associated with inflammation and/or autoimmune disease.

Immunoglobulin single variable domains with high affinity to VEGF are described in, inter alio, WO2008/149147, in particular, the domain antibody DOM15-26-593, which has a polypeptide sequence as set out herein in amino acids 1 to 116 of SEQ ID NO:1. DOM15-26-593 is described in the form of a domain antibody conjugated to a domain of an antibody constant region in WO2008/149150 (an example of which is shown in SEQ ID NO:1 in full). The immunoglobulin single variable domains described in WO2008/149147 and WO2008/149150 are useful candidates in the treatment or prophylaxis of disorders in which VEGF is implicated. The present invention relates to variants of the DOM15-26-593 and DOM15-26-593-Fc molecules described in WO2008/149147 and WO2008/149150. These variants can also be therapeutically efficacious in any of the diseases or conditions described in WO2008/149147 and O2008/149150. In particular, it is envisaged that the immunoglobulin single variable domains, and polypeptides and antigen-binding constructs comprising these can be used in medicine, for example, for the treatment of cancer and/or ocular diseases such as Diabetic Macular Edema, Wet AMD (age-related macular degeneration). Diabetic retinopathy, RVO (retinal vein occlusion) or corneal neovascularisation.

In certain embodiments, the V, domain, polypeptide or antigen-binding construct of the invention are efficacious in treating or ameliorating diseases associated with VEGF signalling when an effective amount is administered. Generally an effective amount is about 1 mg/kg to about 10 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg).

The term “Effector Function” as used herein is meant to refer to one or more of Antibody Dependant Cell mediated Cytotoxic activity (ADCC) , Complement-Dependant Cytotoxic activity (CDC) mediated responses, Fc-mediated phagocytosis and antibody recycling via the FcRn receptor. For IgG antibodies, effector functionalities including ADCC and ADCP are mediated by the interaction of the heavy chain constant region with a family of Fcy receptors present on the surface of immune cells. In humans these include FcyRl (CD64), FcyRll (CD32) and FcyRIII (CD16). Interaction between the antibody bound to antigen and the formation of the Fc/Fcy complex induces a range of effects including cytotoxicity, immune cell activation, phagocytosis and release of inflammatory cytokines.

The interaction between the constant region of an antibody and various Fc receptors (FcR) is believed to mediate the effector functions of the antibody. Significant biological effects can be a consequence of effector functionality, in particular, antibody-dependent cellular cytotoxicity (ADCC), fixation of complement (complement dependent cytotoxicity or CDC), and half-life/clearance of the antibody. Usually, the ability to mediate effector function requires binding of the antigen binding protein to an antigen and not all antigen binding proteins will mediate every effector function.

Effector function can be measured in a number of ways including for example via binding of the FcyRIII to Natural Killer cells or via FcyRl to monocytes/macrophages to measure for ADCC effector function. For example an antigen binding protein of the present invention can be assessed for ADCC effector function in a Natural Killer cell assay. Examples of such assays can be found in Shields et al, 2001 The Journal of Biological Chemistry, Vol. 276, p6591-6604; Chappel et al, 1993 The Journal of Biological Chemistry, Vol 268, p25124-25131; Lazar et al, 2006 PNAS, 103; 4005-4010.Examples of assays to determine CDC function include that described in 1995 J Imm Meth 184:29-38.

Some isotypes of human constant regions, in particular IgG4 and IgG2 isotypes, essentially lack the functions of a) activation of complement by the classical pathway; and b) antibody-dependent cellular cytotoxicity. Various modifications to the heavy chain constant region of antigen binding proteins may be carried out depending on the desired effector property. IgG1 constant regions containing specific mutations have separately been described to reduce binding to Fc receptors and therefore reduce ADCC and CDC (Duncan et al. Nature 1988, 332; 563-564; Lund et al. J. Immunol. 1991, 147; 2657-2662; Chappel et al. PNAS 1991, 88; 9036-9040; Burton and Woof, Adv. Immunol. 1992, 51;1-84; Morgan et al., Immunology 1995, 86; 319-324; Hezareh et al., J. Virol. 2001, 75 (24); 12161-12168).

In one aspect the antigen binding construct comprises, consists of, or consists essentially of, an Fc region of an antibody, or a part thereof, linked to a V_{H} domain according to the invention.

In another aspect the antigen binding construct consists of, or consists essentially of, an Fc region of an antibody, or a part thereof, linked at each end, directly or indirectly (for example, via a linker sequence) to a V_{H} domain according to the invention. Such an antigen binding construct may comprise 2 V_{H} domains separated by an Fc region, or part thereof. By separated is meant that the epitope-binding domains are not directly linked to one another, and in one aspect are located at opposite ends (C and N terminus) of an Fc region, or any other scaffold region. In one embodiment, the antigen binding construct comprises 2 scaffold regions each bound to 2 V_{H} domains, for example at the N and C termini of each scaffold region, either directly or indirectly via a linker.

In another aspect the V_{H} domain is attached to the C terminal end of the Fc region or part thereof, either directly or indirectly by a linker. The construct may be expressed as a fusion protein, or the scaffold and V_{H} domain attached by other means such as chemical conjugation using methods well known in the art.

Such linkers may be the linker “AS” or “GS” or may be one selected from those set out in WO2009/068649, WO2010/136482, PCT/EP2011/070868 or USSN61/512,138, the content of which are incorporated in their entirety.

In an embodiment, the C-terminus of the V_{H} domain is conjugated to a human Fc region. Optionally wherein the N-terminus of the Fc is linked (optionally directly linked) to the C-terminus of the variable domain.

In one embodiment, the immunoglobulin single variable domain or polypeptide in accordance with the invention can be part of a “dual-specific ligand” which refers to a ligand comprising a first antigen or epitope binding site (a first immunoglobulin single variable domain) and a second antigen or epitope binding site (a second immunoglobulin single variable domain), wherein the binding sites or variable domains are capable of binding to two antigens (e.g., different antigens or two copies of the same antigen) or two epitopes on the same antigen which are not normally bound by a monospecific immunoglobulin. For example, the two epitopes may be on the same antigen, but are not the same epitope or sufficiently adjacent to be bound by a monospecific ligand. In one embodiment, dual specific ligands according to the invention are composed of binding sites or variable domains which have different specificities, and do not contain mutually complementary variable domain pairs (i.e. V_{H}/V_{L} pairs) which have the same specificity (i.e., do not form a unitary binding site). Dual-specific ligands and suitable methods for preparing dual-specific ligands are disclosed in WO 2004/058821, WO 2004/003019, and WO 03/002609.

In one embodiment, immunoglobulin single variable domains in accordance with the invention may be used to generate dual or multi-specific compositions or fusion polypeptides (herein “antigen-binding constructs”). Accordingly, immunoglobulin single variable domains in accordance with the invention may be used in larger constructs. Suitable constructs include fusion proteins between an anti-SA immunoglobulin single variable domain (dAb) and a monoclonal antibody, synthetic pharmaceutical (small molecule, NCE), protein or polypeptide and so forth. Accordingly, anti-SA immunoglobulin single variable domains in accordance with the invention may be used to construct multi-specific molecules, for example, bi-specific molecules such as dAb-dAb (i.e. two linked immunoglobulin single variable domains in which one is an anti-SA dAb), mAb-dAb or polypeptide-dAb constructs. In these constructs the anti-SA dAb (AlbudAb™) component provides for half-life extension through binding to serum albumin (SA). Suitable mAb-dAbs and methods for generating these constructs are described, for example, in WO2009/068649.

The immunoglobulin single variable (V_{H}) domains of the invention and polypeptides comprising these can also be formatted to have a larger hydrodynamic size, for example, by attachment of a PEG group, serum albumin, transferrin, transferrin receptor or at least the transferrin-binding portion thereof, an antibody Fc region, or by conjugation to an antibody domain.

Hydrodynamic size of the V_{H} domains of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g., the size of a PEG moiety attached to a dAb monomer), can be varied depending on the desired application. For example, where ligand is intended to leave the circulation and enter into peripheral tissues, it is desirable to keep the hydrodynamic size of the ligand low to facilitate extravazation from the blood stream. Alternatively, where it is desired to have the ligand remain in the systemic circulation for a longer period of time the size of the ligand can be increased, for example by formatting as an Ig like protein.

V_{H} domains of the invention can also be conjugated or linked to an anti-serum albumin or anti-neonatal Fc receptor antibody or antibody fragment, e.g. an anti-SA or anti-neonatal Fc receptor dAb, Fab, Fab′ or scFv, or to an anti-SA affibody or anti-neonatal Fc receptor affibody.

WO04/003019 and WO2008/096158 disclose anti-serum albumin (SA) binding moieties, such as anti-SA immunoglobulin single variable domains (dAbs), which have therapeutically-useful half-lives. These documents disclose monomer anti-SA dAbs as well as multi-specific ligands comprising such dAbs, e.g., ligands comprising an anti-SA dAb and a dAb that specifically binds a target antigen, such as TNFR1. Binding moieties are disclosed that specifically bind serum albumins from more than one species, e.g. human/mouse cross-reactive anti-SA dAbs.

WO05/118642 and WO2006/059106 disclose the concept of conjugating or associating an anti-SA binding moiety, such as an anti-SA immunoglobulin single variable domain, to a drug, in order to increase the half-life of the drug. Protein, peptide and new chemical entity (NCE) drugs are disclosed and exemplified. WO2006/059106 discloses the use of this concept to increase the half-life of insulintropic agents, e.g., incretin hormones such as glucagon-like peptide (GLP)-1.

Reference is also made to Holt et al, “Anti-Serum albumin domain antibodies for extending the half-lives of short lived drugs”, Protein Engineering, Design & Selection, vol 21, no 5, pp283-288, 2008.

The invention also provides isolated and/or recombinant nucleic acid molecules encoding polypeptide (single variable domains, fusion proteins, polypeptides, dual-specific ligands and multispecific ligands) as described herein.

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g, plasmids, phagemids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g, promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic (e.g, lac, tac, T3, T7 promoters for E. coli) and eukaryotic (e.g, Simian Virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in prokaryotic (e.g., lactamase gene (ampicillin resistance), Tet gene for tetracycline resistance) and eukaryotic cells (e.g, neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g, LEU2, URA3, H153) are often used as selectable markers in yeast. Use of viral (e.g, baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acad. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NSO cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In certain embodiments, the host cell is a non-human host cell. In a particular embodiment, the host cell is a CHO cell.

In one embodiment, the polypeptides or immunoglobulin single variable domains in accordance with the invention are secreted when expressed in a suitable expression system, optionally a CHO cell based expression system. Suitably, the amino acid substitutions in accordance with the invention do not lead to loss of expression. Suitably, the amino acid substitutions in accordance with the invention lead to enhancement of expression (i.e. increased titre).

Additional expression systems include cell free systems. In yet another embodiment, expression of variable domains can be accomplished using cell-free expression systems such as those described in WO2006/018650 and WO2006/046042.

Reference is made to WO200708515, page 161, line 24 to page 189, line 10 for details of disclosure that is applicable to embodiments of the present invention. This disclosure is hereby incorporated herein by reference as though it appears explicitly in the text of the present disclosure and relates to the embodiments of the present invention, and to provide explicit support for disclosure to incorporate into claims below. This includes disclosure presented in WO200708515, page 161, line 24 to page 189, line 10 providing details of the “Preparation of Immunoglobulin Based Ligands”, “Library vector systems”, “Library Construction”, “Combining Single Variable Domains”, “Characterisation of Ligands”, “Therapeutic and diagnostic compositions and uses”, as well as definitions of “operably linked”, “naive”, “prevention”, “suppression”, “treatment”, “therapeutically-effective dose” and “effective”.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Ka bat numbering convention. For further information, see Ka bat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Mini- |
|---|---|---|---|---|---|
| | | | | | mum |
| | Kabat | Chothia | AbM | Contact | binding |
| | CDR | CDR | CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |

The invention will be described further with reference to the following examples. The objective of these examples was to improve the biophysical characteristics of an anti-VEGF dAb-Fc construct and thus ease downstream processing. Although the data presented here is in the dAb-Fc format, these observations should be valid for naked dAbs or dAbs in different formats.

Methods:

SEC and SEC MALLS (size exclusion chromatography with multi-angle-LASER-light-scattering) is a non-invasive technique for the characterisation of macromolecules in solution. SEC separates proteins by molecular size and is able to differentiate between the monomeric state and aggregated states such as dimer, trimer etc. Aggregates may be reversible or irreversible, covalent or non-covalently linked, and involve specific or non-specific interactions. Defined aggregates may include but are not limited to dimers, trimers etc. The level of aggregation and hence %monomeric protein may be dependent upon the conditions experienced by the protein such as pH, temperature, buffer, protein concentration. Following separation, the propensity of the protein to scatter light may be measured using a multi-angle-LASER-light-scattering (MALLS) detector (Wyatt, US).

Differential scanning calorimetry (DSC) is a thermoanalytical technique in which the difference in the amount of heat required to increase the temperature of a sample and reference are measured as a function of temperature. It can be used to study a wide range of thermal transitions in proteins and is useful for determining the melting temperatures as well as thermodynamic parameters.

Analytical Ultra-Centrifugation (AUC): Sedimentation equilibrium is a method for measuring solution molecular mass (described, for example, in Lebowitz et al. Protein Science (2002), 11:2067-2079).

### EXAMPLE 1

### Surface exposure and Analysis of a anti-VEGF Vu dAb

In an attempt to increase solubility of the VEGF dAb-Fc, DOM15-26-593-Fc (SEQ ID NO:1), and thus decrease the propensity of the molecule to aggregate/precipitate, the V_{H} domain residues were assessed for surface exposure and hydrophobicity.

A homology model of the αVEGF dAb [DOM15-26-593] was generated in Accelrys Discovery Studio using domain antibody 10HQ (Jespers et al. ibid.) as a template. Solvent accessibility was calculated from the structure and plotted across the sequence, together with hydrophobicity. Analysis of the plot and the structure identified Tyr56 and Tyr58 as solvent exposed hydrophobic residues that could potentially be involved in aggregation. Additionally, aggregation potential was calculated across the sequence and combined with the solvent accessibility scores by normalising both scores between 0 and 1 and then averaging or multiplying the two resultant scores. Plotting the final scores against the sequence again identified Tyr56 and Tyr58 as potential aggregation-prone residues, Tyr56 being especially prominent. The frequency of different amino acids at these two positions was calculated, both for human germline V_{H} genes, and for all human V regions sequence extracted from the NCB! IgSeq database. Residues were identified that were found at high levels in naturally occurring monoclonal antibody V regions and their hydrophobicity levels analysed. The hydrophilic residue, asparagine, was identified as the second most common residue at position 56 (˜15%) behind serine (˜40%), and the second most common residue at position 58 (˜22%) behind tyrosine (˜45%), and was thus chosen for potential back-mutations to ameliorate the observed aggregation.

| DOM15-26-593 V_{H} domain | |
|---|---|
| (SEQ ID NO: 1) | |
| 1 | EVQLLVSGGG LVQPGGSLRL SCAASGFTFK AYPMMWVRQA PGKGLEWVSE ISPSGSYTYY |
| 60 | ADSVKGRFTI SRDNSKNTLY LQMNSLRAED TAVYYCAKDP RKLDYWGQGT LVTVS |

The leucine residues at positions 5 and 108 (underlined above) were identified as surface-exposed and therefore candidates for possible hydrophobic to hydrophilic mutations. A further 3 hydrophobic residues were identified within human germline V regions—a glycine residue at position 44 and two tyrosine residues within CDR2 (also underlined above).

The following mutations to hydrophilic amino-acids were chosen following analysis of naturally-occurring human germline mutations in V regions: L50, G44R, Y56N, Y58N, L108T. Residue 56 is within the second CDR region, as is residue 58 (under some definitions) and therefore mutations to these residues may impact upon binding/specificity.

### EXAMPLE 2

### Creation of Variant dAb Molecules

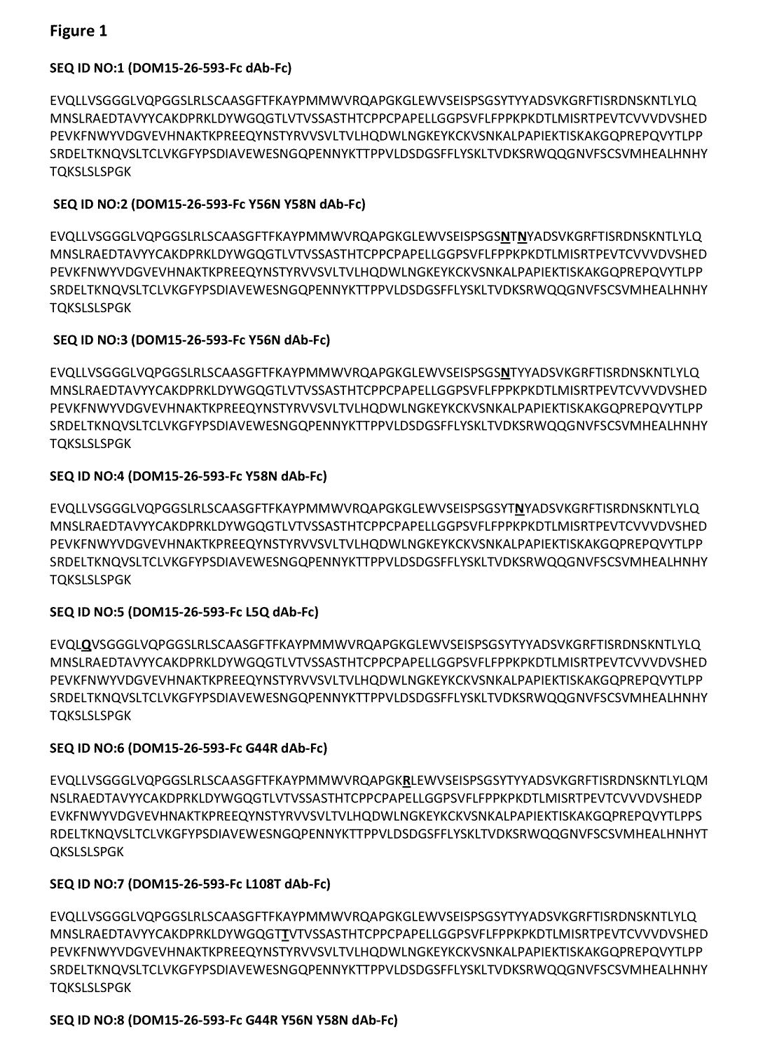

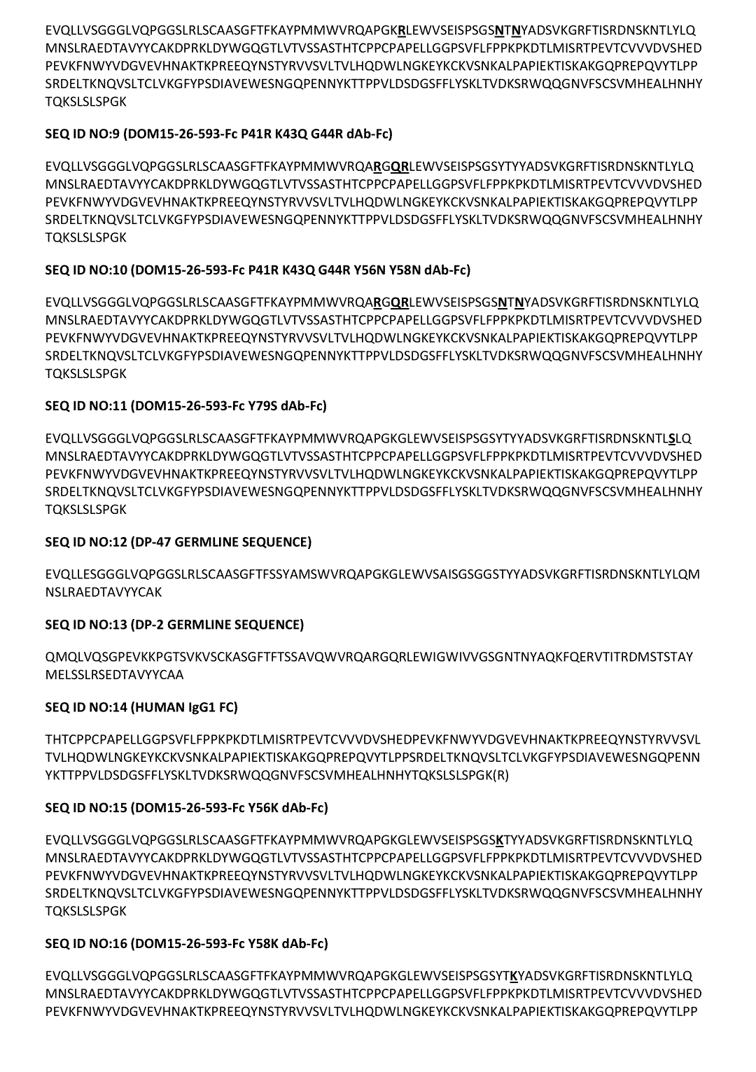

The following variant V_{H} regions (in the form of V_{H} dAb-Fc molecules) were generated by site directed mutagenesis in the DOM15-26-593-Fc molecule of SEQ ID NO:1 (sequences shown in FIG. 1):
1. Y56N & Y58N (SEQ ID NO:2)
2. L5Q (SEQ ID NO:5)
3. G44R (SEQ ID NO:6)
4. L108T (SEQ ID NO:7)

The parental (SEQ ID NO:1) and variant constructs were then sub-cloned into a CHROMOS ATV vector (for a description of the CHROMOS system see Lindenbaum et al, NAR, 32, e172, 2004) and CHROMOS pooled (or bulk) transfections and subsequently single cell clones thereof were generated for each plasmid. Expression, binding and aggregation were assessed for each. The CHROMOS system is a site-specific integration system, whereby expression cassettes are site-specifically integrated onto an artificial chromosome present within the host cell. Using this system enables the direct comparison of expression levels of differing molecules since there are no ‘position-effects’ such as those associated with random genomic integration.

### EXAMPLE 3

### Biophysical and Functional Analysis of Variant dAb Molecules

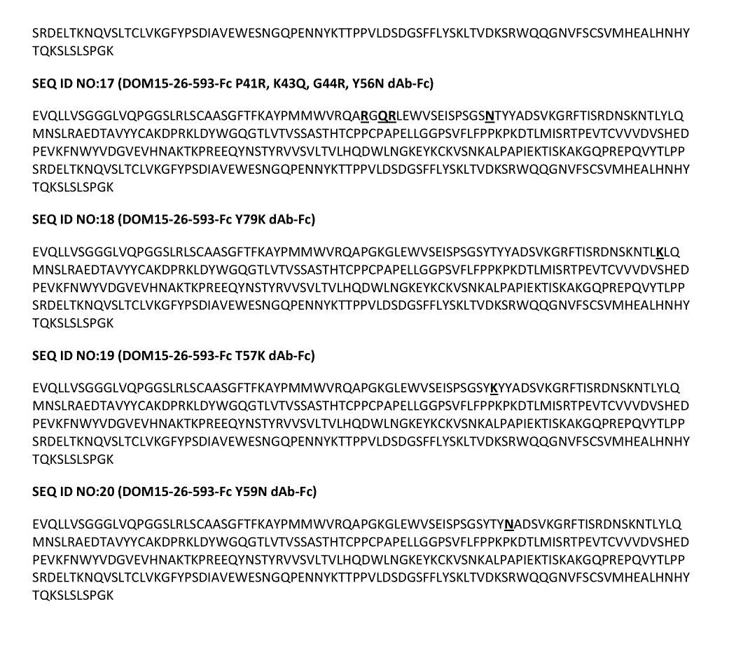

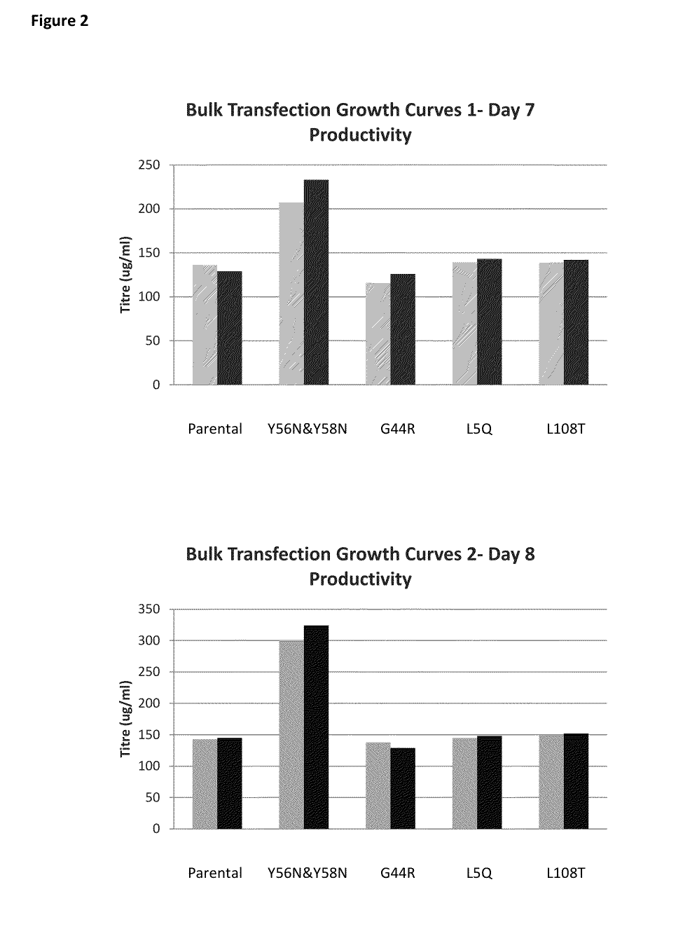

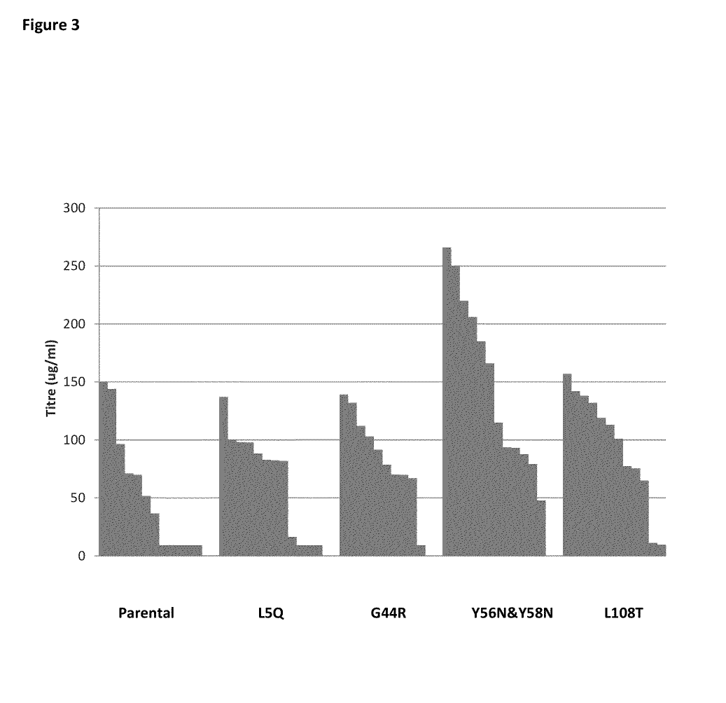

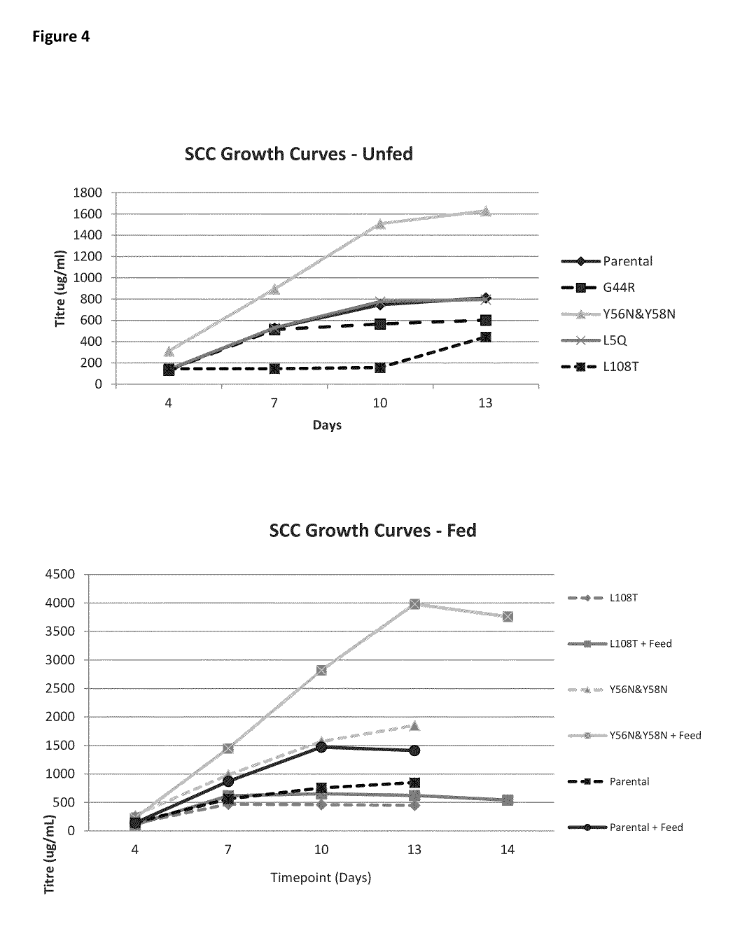

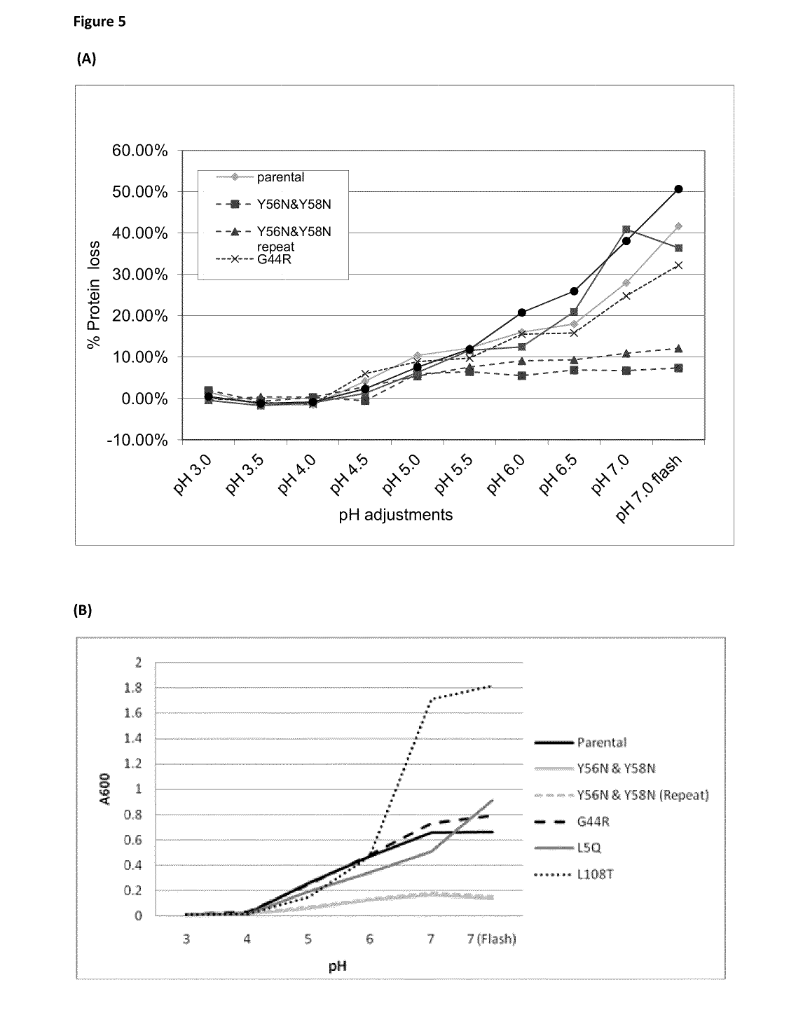

The DOM15-26-593-Fc Y56N&Y58N variant of SEQ ID NO:2 (also referred to herein as the “CDR2 variant” was surprisingly found to have increased expression (2-3-fold) over wild type (FIGS. 2-4). Binding was only mildly affected (FIG. 5E) and binding kinetics (by BIACORE®) showed the dissociation constant (kd) was affected. The rest of the variants (G44R, L5Q and L108T) showed similar expression and binding to the parental molecule.

The Y56N&Y58N variant was found to have decreased aggregation/precipitation properties as compared to the parental, whilst the L108T mutation was found to increase precipitation (FIG. 5A-C). The remaining variants - L5Q and G44R behaved more similarly to the parental DOM15-26-593-Fc molecule, although slight improvements in biophysical characteristics were observed for the G44R mutation (FIG. 5C).

The variants were designed to improve the hydrophilicity of the surface of the molecule. Residues were selected for mutation based on their hydrophobicity and their surface accessibility. The most successful mutations were those at positions 56 and 58, where a tyrosine residue was substituted with an asparagine residue at both positions. This should reduce the hydrophobicity of the surface of the molecule by the removal of the hydrophobic tyrosine residue and replacement with the more polar asparagine residue.

Reduction in hydrophobicity is supported by downstream processing (DSP) data where reduced column volumes were required for elution from a Protein A column for the DOM15-26-593-Fc Y56N Y58N variant molecule (SEQ ID NO:2) compared to the YTY motif in the parental molecule (SEQ ID NO:1)—a 2 fold difference. This suggests that non-specific binding, often due to exposure of hydrophobic patches, is reduced in the variant molecule.

Upon titration from pH 3 to pH 7, precipitation was observed with the parental molecule. Upon a similar titration with the DOM15-26-593-Fc Y56N Y58N variant less precipitation was observed (˜10% protein loss compared to WT 40% at pH 7, FIG. 5A) suggesting an improvement in the biophysical properties of the molecule. This was supported by A600 absorbance data as the pH increased for the DOM15-26-593-Fc Y56N Y58N variant compared to the parent (FIG. 5B). A600 monitors the light scattered by particulates in the solution and can help to quantify visual observations, as well as picking up small levels of particles not visible to the naked eye. Furthermore, a loss in soluble protein was observed as the pH increased, correlating with the increased precipitation and A600 values, suggesting that the precipitant is made up of protein.

SEC data suggests that the soluble protein remaining is largely monomeric, with few dimers or small oligomers at any pH (except pH 3) (FIG. 5C). DLS data, which reports on higher order oligomers or aggregates, suggests an increase in large size aggregates as the pH increases (it is likely that these are too big to enter an SEC column, and therefore would not be observed). This suggests that the precipitation observed is as a result of aggregation. It appears that a stable dimer is not formed as an intermediate in this process.

No reduction in the melting temperature observed between the different mutants (WT, CDR2 and

L108T shown in FIG. 5D) suggests that incorporation of surface mutations does not impact the conformational fold of the molecule or decrease its conformational stability. An increase in expression levels often correlates with an increase in conformational stability. In this case the increase in expression (FIGS. 2-4) cannot be attributed to increased conformational stability, instead a possible explanation is a reduction in the level of aggregated/misfolded species in the cell as a result of a less hydrophobic surface, meaning that the protein is better tolerated.

Surprisingly, the L108T mutant showed worsened behaviour compared to wt (FIGS. 5A-C). L50 was very similar to WT and G44R possibly showed a minor improvement (see SEC-HPLC data in FIG. 5C).

### EXAMPLE 4

### Creation of a Second Set of Variant dAb Molecules

To determine whether the Y56N or Y58N substitution alone might be sufficient for the molecule to retain the favourable biophysical properties without the small compromise in binding it was decided to substitute the two tyrosine amino-acids present within CDRH2 individually. To investigate the effects of the G44R mutation further it was also decided to combine this substitution with the Y56N Y58N substitutions.

In addition we observed that both the CDR2 and G44R (found in framework 2) mutations were found to be naturally occurring in the human germline V_{H} DP-2 sequence, whilst the parental DOM15-26-593-Fc is based upon the human germline DP-47 sequence. Analysis of the framework 2 sequences in both germline sequences revealed two further differences at positions 41 and 43, underlined below:

DP-2 Framework 2
WVRQARGQRLEWIG
DP-47 Framework 2
WVRQAPGKGLEWVS

To essentially alter framework 2 of the DOM15-26-593-Fc to that of DP-2, the G44R substitution was combined with P41R (hydrophobic to hydrophilic) and K43Q (both hydrophilic) substitutions. Furthermore, the resulting ‘framework 2’ variant was also combined with the Y56N Y58N substitutions.

Finally, in silico analysis also highlighted a tyrosine residue at position 79 (within framework 3). In both the DP-2 and DP-47 sequences this tyrosine is conserved, however in other human germline V_{H} sequences valine or serine residues are found at this position. A sixth variant containing a Y79S substitution was also generated, to see whether a mutation in this framework might impact molecule aggregation/precipitation.

The six new variants that were generated are therefore as follows:
1. Y56N (SEQ ID NO:3)
2. Y58N (SEQ ID NO:4)
3. G44R & Y56N & Y58N (SEQ ID NO:8)
4. P41R & K43Q & G44R (SEQ ID NO:9)
5. P41R & K43Q & G44R & Y56N & Y58N (SEQ ID NO:10)
6. Y79S (SEQ ID NO:11)

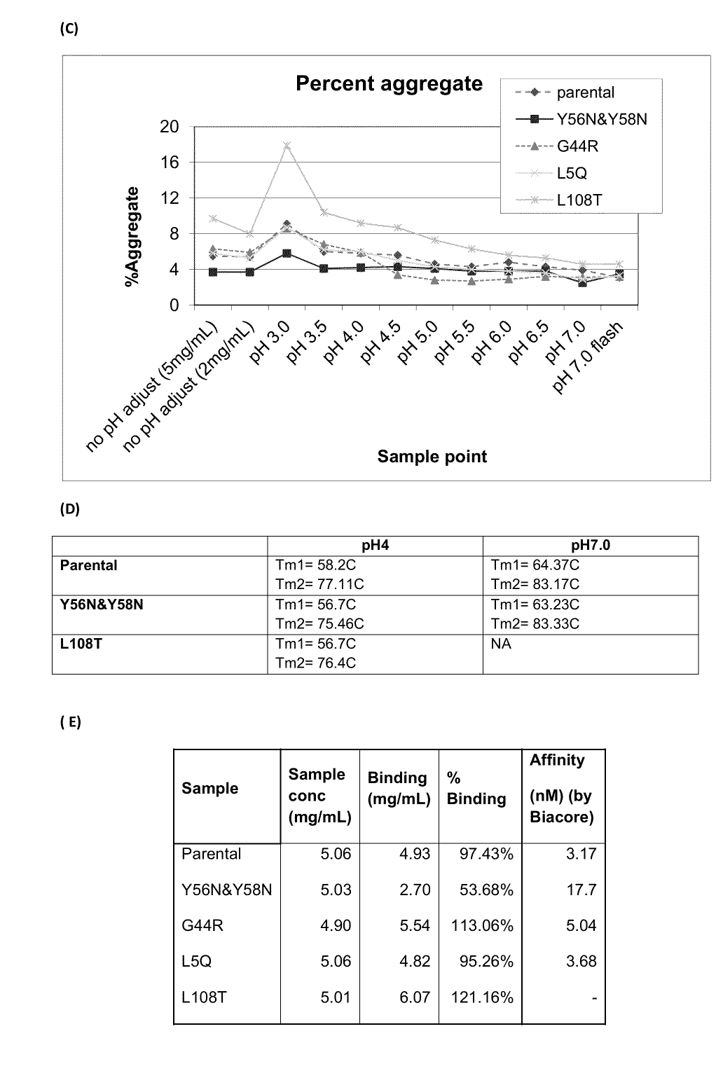

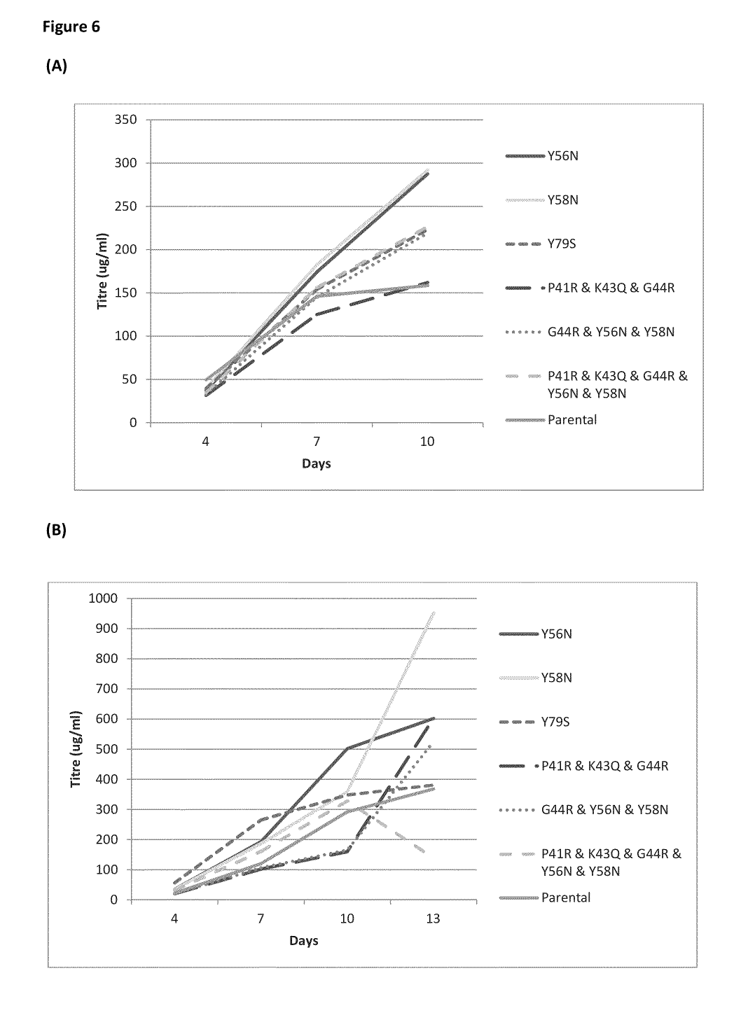

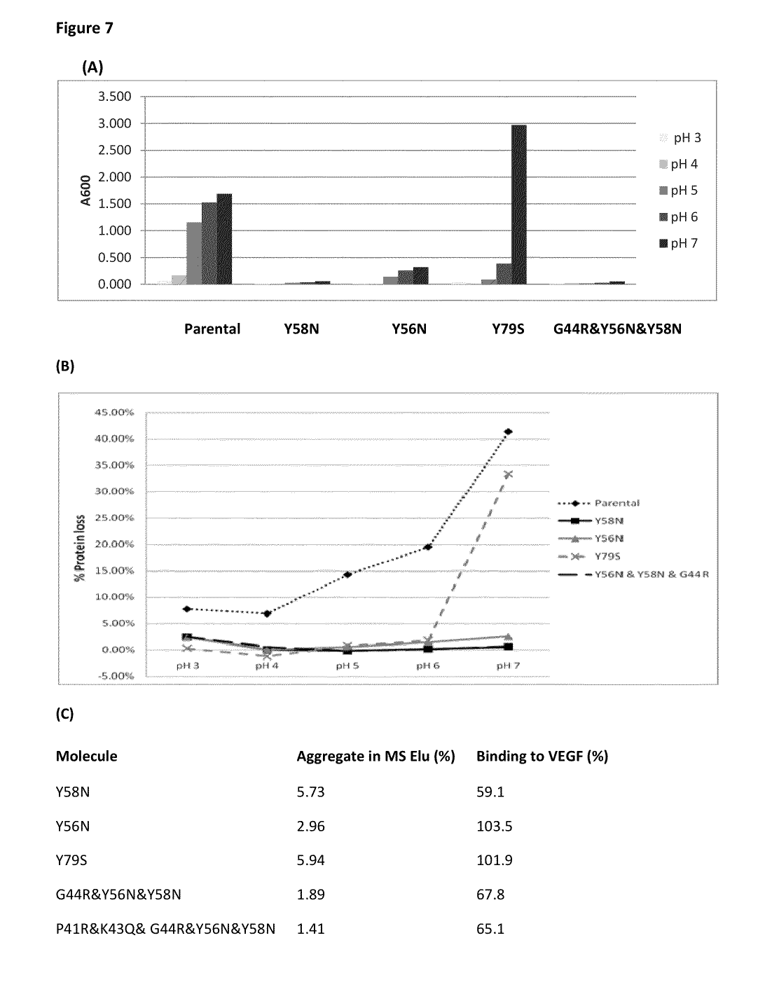

The resulting data obtained showed that both the Y56N and the Y58N variants retained higher expression than the parent (FIG. 6) and the improved biophysical properties the CDR2 mutant had (FIG. 7), however, In the case of this dAb-Fc molecule it was only the Y56N variant that fully retained binding.

pH 7.0 solubility of the purified variants was achieved in all cases, except Y79S (FIG. 7B). Binding ELISA data on MS eluate pH 4 indicates 60% activity for the Y58N variant, and the G44R & Y56N & Y58N variant versus 100% activity for Y56N and Y79S (FIG. 7C). The Y56N was therefore chosen for further studies as it fulfilled binding and solubility criteria with minimal product loss during chromatography & pH adjustment steps.

Interestingly, the Y79S mutant showed improved performance at lower pHs but this declined at pH 7 (FIG. 7). Y56N & Y58N, when combined with P41R & K43Q & G44R also showed improved properties (FIG. 7).

### EXAMPLE 5

### Biophysical Characterisation of the Y56N Mutant in Comparison to the Wild Type (WT) Molecule

The Y56N mutant and wild type (WT) molecule were further characterised over a wide range of pH, buffer and buffer strength conditions that might be experienced within downstream processing or formulation.

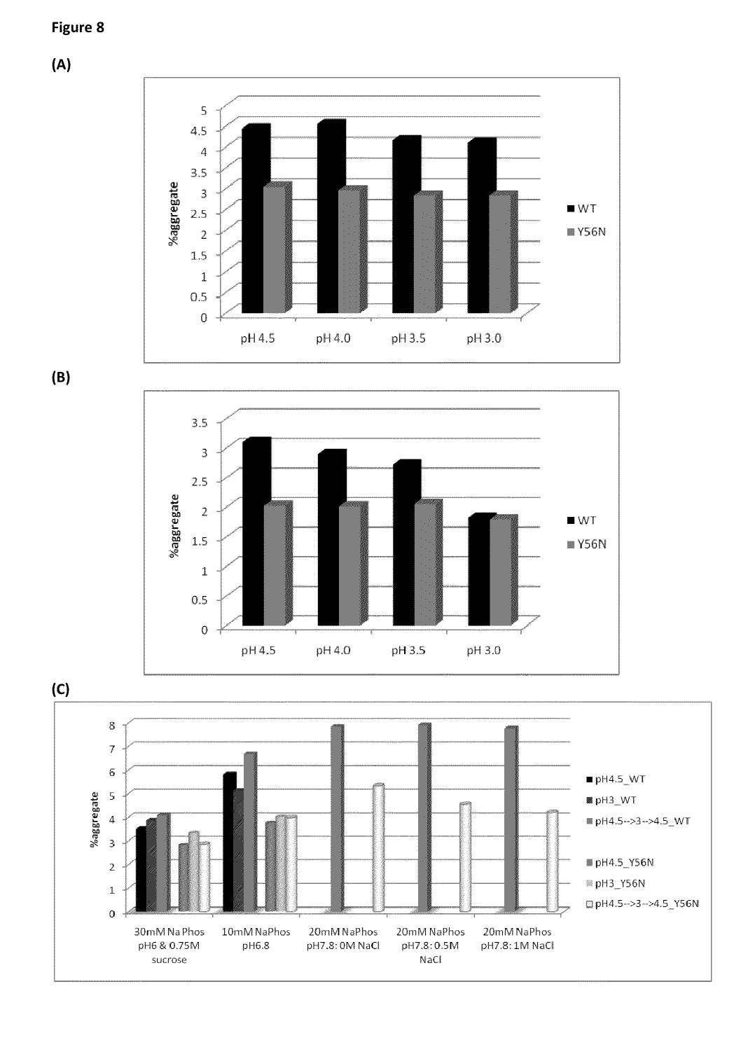

Biophysical analysis was carried out on samples at low pH that might be experienced during elution from affinity columns, such as citrate or acetate buffer between pH3.0 and 4.5. The primary differences between the two molecules were observed in the SEC-HPLC data, which showed a decrease in the % aggregate observed for the Y56N mutant compared to the WT in both the 25 mM sodium citrate and 30mM sodium acetate buffers at all the pH's measured (FIG. 8A and B). In sodium acetate buffer the WT displayed sensitivity to pH with aggregate levels increasing with increasing pH, which was not observed for the Y56N variant.

Samples in 100 mM sodium acetate buffer at pH3, pH4.5 or pH4.5 adjusted to pH3 and then back to pH4.5 (labelled 4.54344.5) were then dialysed into 30mM sodium phosphate pH6, 0.75M sucrose buffer and assessed in three different salt concentrations (0, 0.1 and 0.2M NaCl). The Y56N mutant showed a reduced aggregate content by SEC-HPLC as compared to that of the WT (FIG. 8C). No effect of NaCl was visible. The WT molecule also demonstrated evidence of fragmentation (˜1%) at pH 3.0. No fragmentation was observed in the Y56N mutant molecule.

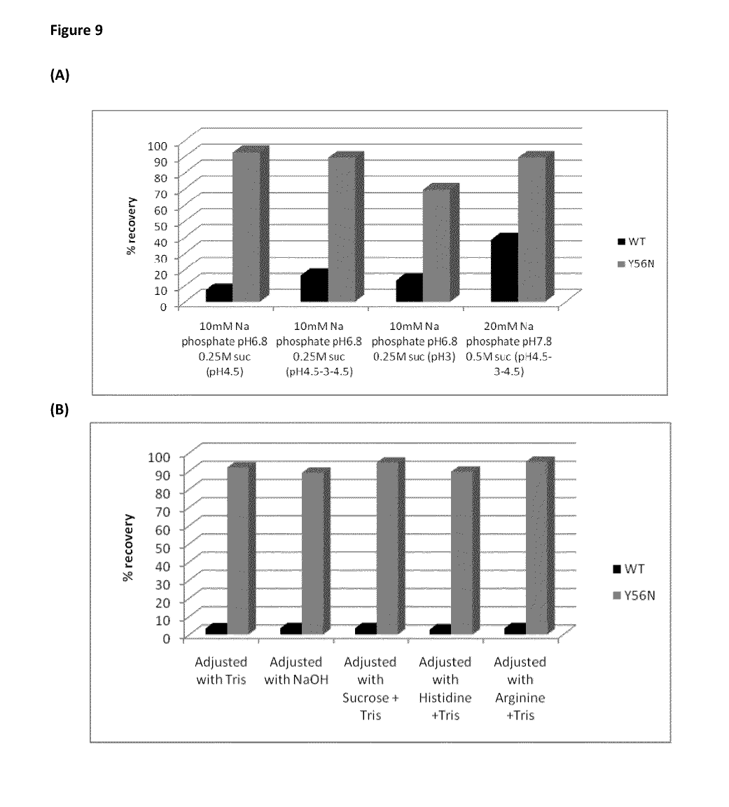

The sample in 30 mM sodium phosphate, pH6, 0.75M sucrose was then adjusted by dilution and pH adjustment to either 10 mM sodium phosphate, 0.25M sucrose, pH 6.8 or 20 mM sodium phosphate, 0.5M sucrose, pH 7.8 followed by concentration of the samples to ˜8 mg/ml. Samples were again analysed in a range of NaCl concentrations (at 0, 0.5 and 1M NaCl). Both the molecules demonstrated precipitation at this step, which was not seen at lower pH values. Severe and continuous precipitation was observed for all the WT samples, leading to very low recoveries compared to the Y56N mutant (FIG. 9A). Precipitation in the case of the Y56N mutant molecule was much less severe as compared to the WT, with no precipitation seen at all in the 20 mM sodium phosphate pH7.8 sample of the Y56N mutant. The remaining soluble material for all Y56N samples was un-aggregated by DLS unlike the WT, which showed the presence of very large particles (>70 nm). SEC-HPLC analysis on the soluble material showed similar trends as seen in FIGS. 8A and B, where the WT showed an increase in aggregation compared with Y56N (FIG. 8C).

Samples of both WT and Y56N in sodium acetate at pH4.5 were also titrated to pH7.5 using a variety of buffers. During pH adjustment severe and continuous precipitation was observed for the WT molecules with maximum solubility being <1 mg/ml. The Y56N mutant showed much improved solubility over the WT, leading to massive increases in recoveries of >85% (FIG. 9B).

In summary, in all the conditions tested, the Y56N mutant demonstrated less aggregation than the WT molecule. The solubility above pH 6 was very poor for the WT as compared to the mutant and overall the WT is more sensitive to pH changes which have negative effects on the molecule when compared with the mutant.

### EXAMPLE 6

### Creation of a Third Set of Variant dAb Molecules

A third series of variants were designed to further investigate the impact of amino-acid mutations upon the biophysical properties of the DOM15-26-593-Fc molecule.

Firstly, to confirm that the improved expression profiles and biophysical characteristics of the Y56N and Y58N variants were due to the mutations from hydrophobic to hydrophilic residues, we designed variants with lysine residues at both of these positions. The Y58K variant is naturally observed within human germline V_{H} regions. Variants containing lysine residues at these positions might also be preferable over asparagine residues, since the latter have the potential to deamidate.

In addition, to investigate any potential additive effects upon expression, the DP2 framework substitutions (P41R & K430 & G44R) and Y56N mutations were combined.

Increased solubility of the Y79S mutant, as compared to wild-type, was observed between pH 3-6 FIG. 7), however the molecule was shown to precipitate at pH7. A variant with increased hydrophilicity, Y79K, was therefore designed to investigate any potential further improvements upon molecule solubility.

Two further variants, T57K and Y59N, were also designed following the further bioinformatics analysis of the wild-type and mutant molecules, having been identified as residues potentially involved in aggregation. The first variant is naturally occurring in human germline V_{H} regions.

This third series of variant molecules generated are therefore as follows:
1. Y56K (SEQ ID NO:15)
2. Y58K (SEQ ID NO:16)
3. P41R, K43Q, G44R, Y56N (SEQ ID NO:17)
4. Y79K (SEQ ID NO:18)
5. T57K (SEQ ID NO:19)
6. Y59N (SEQ ID NO:20)

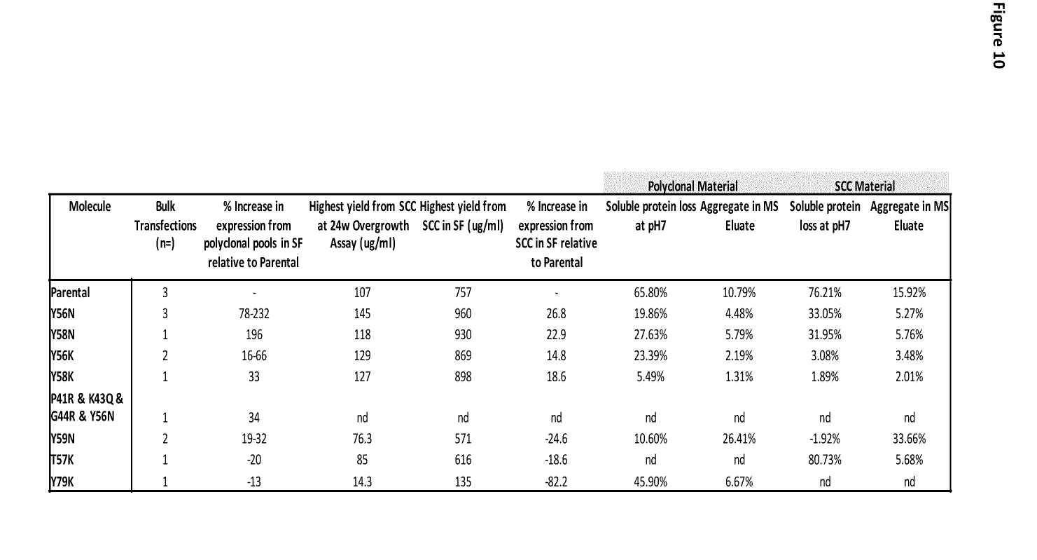

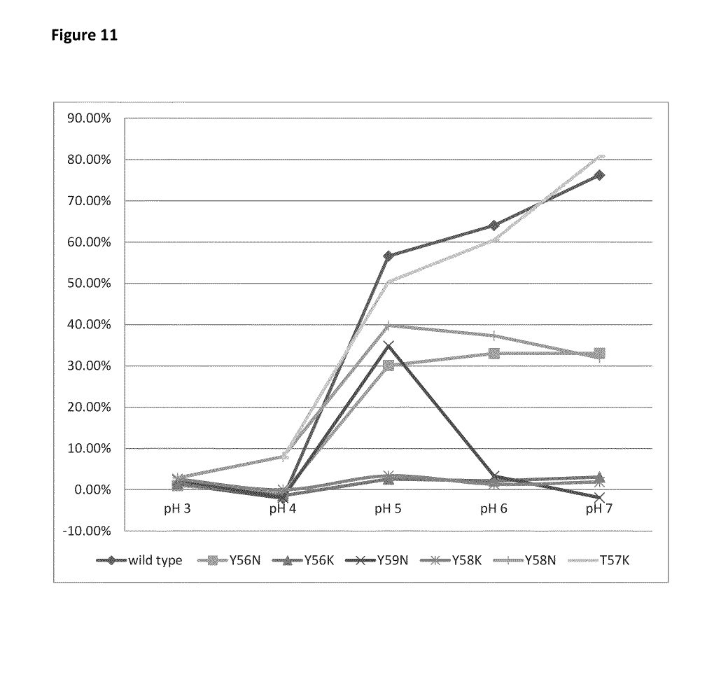

The Y56K and Y58K variants both exhibited higher expression than the parental molecule (FIG. 10), furthermore both molecules were found to have improved biophysical properties as compared to the parental molecule and the Y56N and Y58N variants (FIGS. 10-11). The P41R, K43Q, G44R, Y56N molecule also exhibited increased expression in polyclonal pools relative to the parental molecule however, as this was significantly lower than for the Y56N mutant alone, the variant was not analysed further (FIG. 10).

The Y59N variant exhibited decrease expression levels relative to parent following single-cell cloning; this molecule demonstrated improved solubility between pH 3-7 however, although an increase in protein loss was observed at pH 5 (FIGS. 10-11).

Previous data for the Y79S mutant showed improved solubility at lower pH as compared to the parental molecule (FIG. 8). Mutation of the same residue to a lysine (Y79K) demonstrated no improvement in expression levels, but a reduction in aggregate levels and improvement in solubility at pH 7 (FIG. 10).

The T57K variant exhibited decrease in expression, no improvement in solubility, but a reduction in aggregate levels (FIG. 10).

No further improvements in either expression or biophysical properties were observed for the remaining variants tested (FIGS. 10-11).

### EXAMPLE 7

### Creation of Three Sets of Variant dAb Molecules with Mutations at Positions 56 or 58

To determine whether the improved expression and biophysical properties observed for the Y56N and Y58N mutants of the DOM15-26-593-Fc molecule could be extrapolated to other dAb-Fc constructs, three sets of variant molecules with mutations to asparagines at these positions were created. Two of the three parent molecules were found to harbour hydrophobic residues, a glycine and a tryptophan, at position 56, whilst the third contains an arginine at this position. All three parent molecules have a tyrosine residue at position 58.

The three sets of variant molecules, including parental, are therefore as follows:

| | | |
|---|---|---|
| 1a.) 15-8 wt | 2a.) 7r-29 wt | 3a.) 21-23 wt |
| 1b.) 15-8 R56N | 2b.) 7r-29 W56N | 3b.) 21-23 G56N |
| 1c.) 15-8 Y58N | 2c.) 7r-29 Y58N | 3c.) 21-23 Y58N |

As for the DOM15-26-593-Fc molecule and variants, all three sets of molecules were transfected in bulk using the CHROMOS system and productivity assessed in shake flasks in polyclonal pools. The biophysical properties of the resulting dAb-Fc molecules were also assessed at this stage. Following the initial productivity screen the polyclonal pools were also single-cell cloned and expression levels compared using a 24-well overgrowth assay.

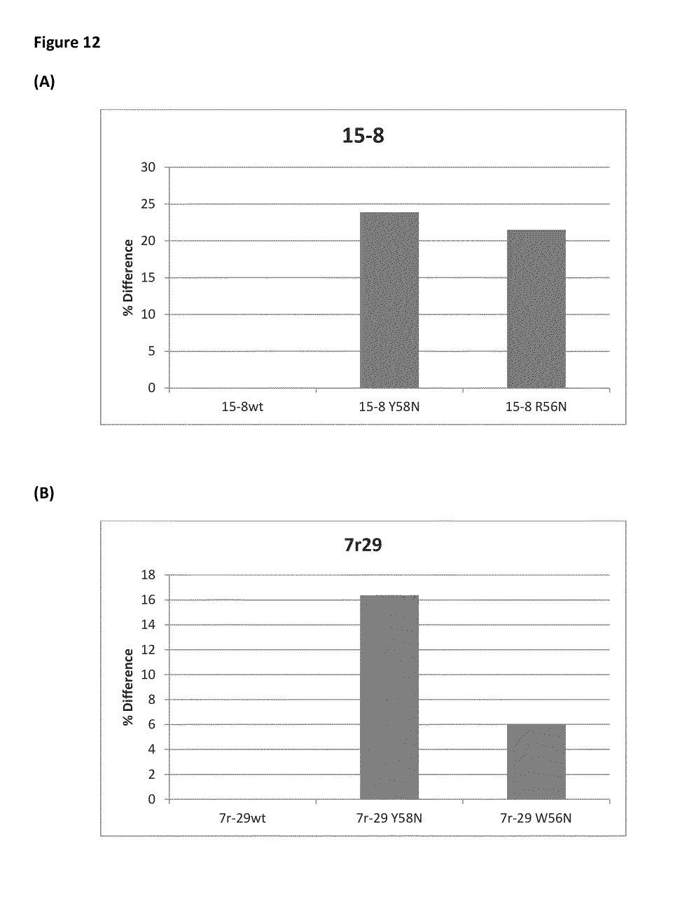

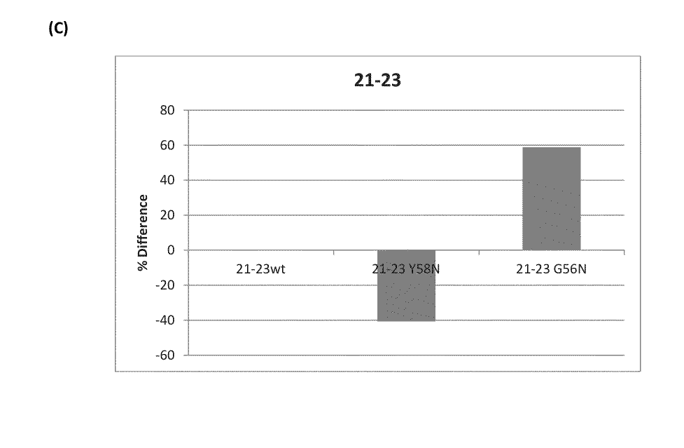

As shown in FIG. 12A and B, the Y58N mutation increased expression of both the 15-8 and 7r-29 molecules in polyclonal pools relative to the parental molecule. For all three dAb-Fc molecules, mutation of the amino acids at position 56 to asparagines was also observed to increase expression relative to parent (FIG. 12 A-C). For the 21-23 molecule however, the Y58N mutant exhibited decreased expression relative to parent (FIG. 12 C).

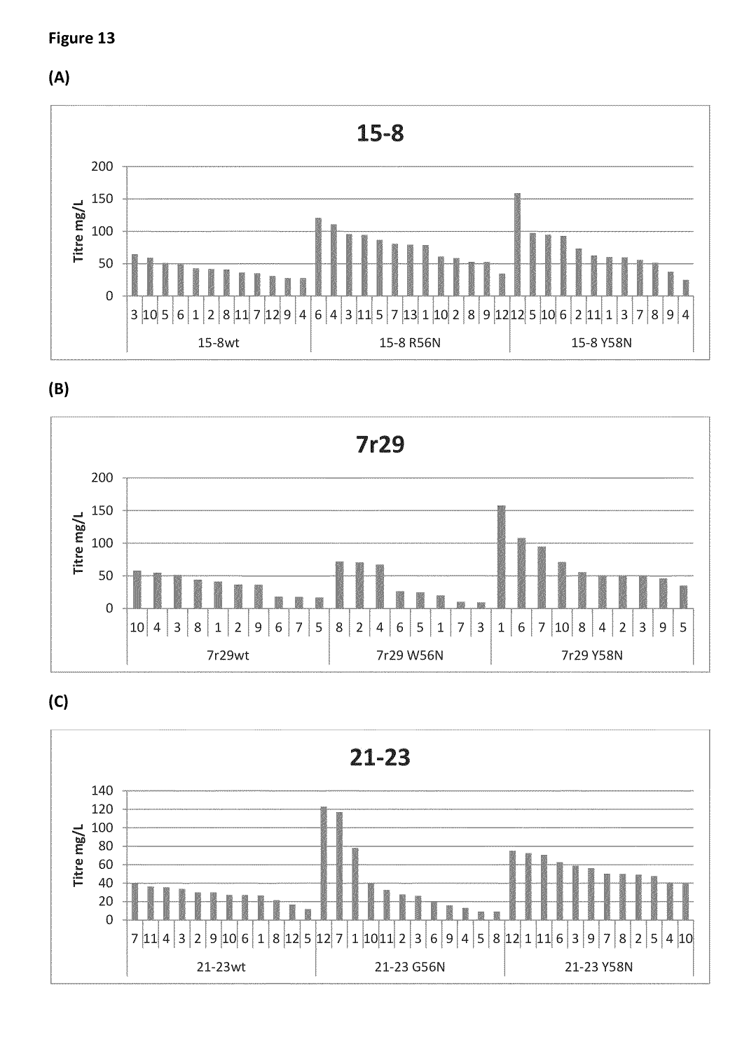

Following single-cell cloning, all of the mutant dAb-Fc molecules were found to express at higher levels than their respective parental molecules in a 24-well overgrowth assay (FIG. 13 A-C).

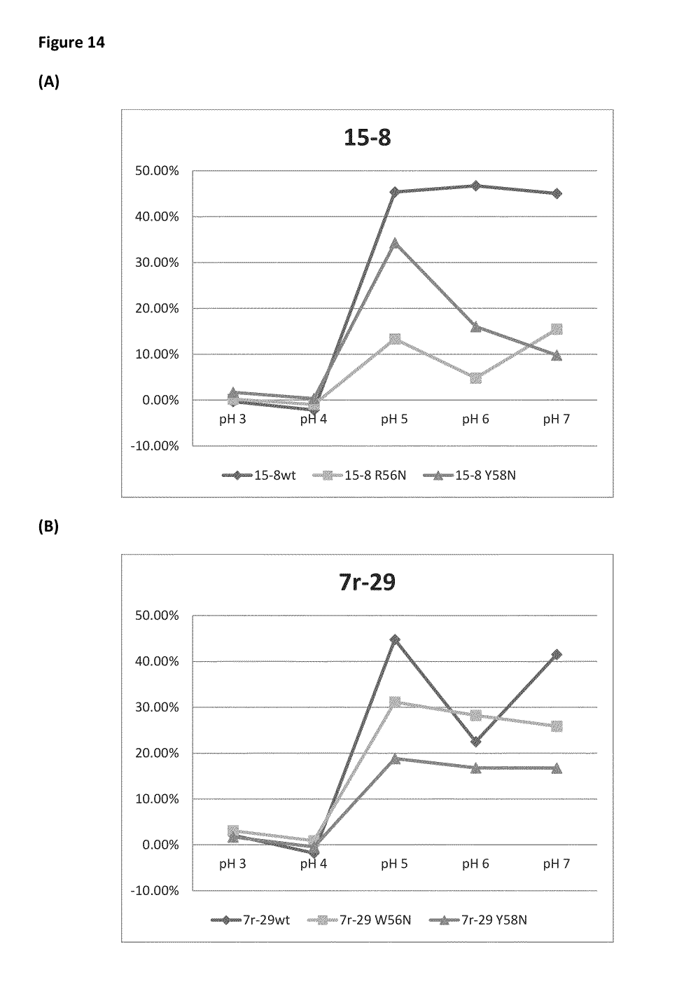

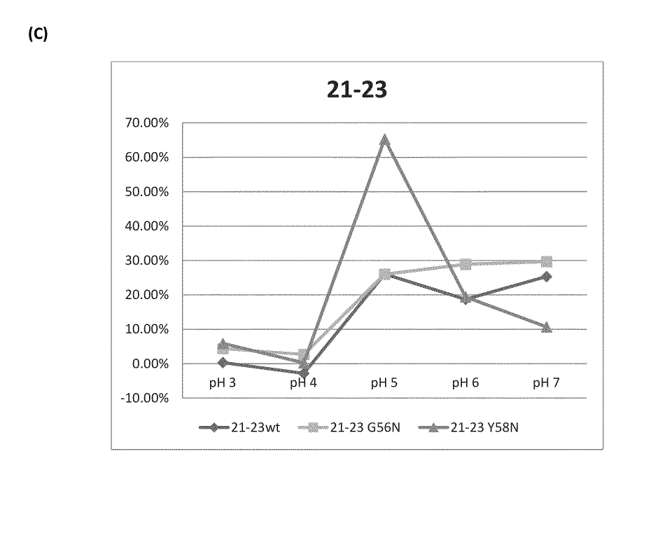

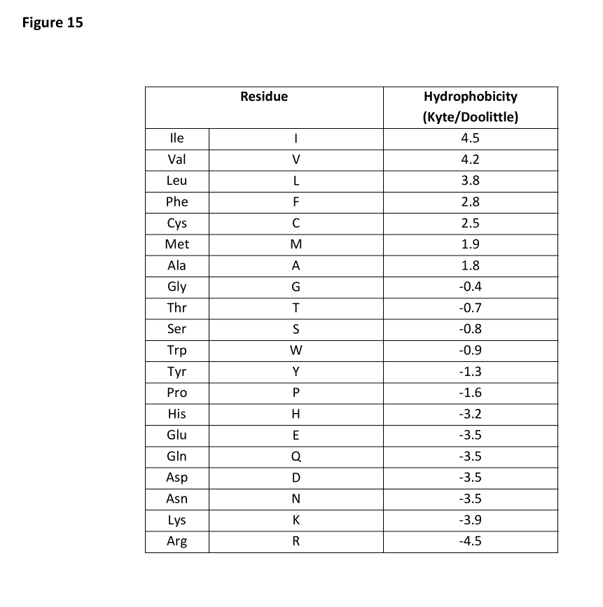

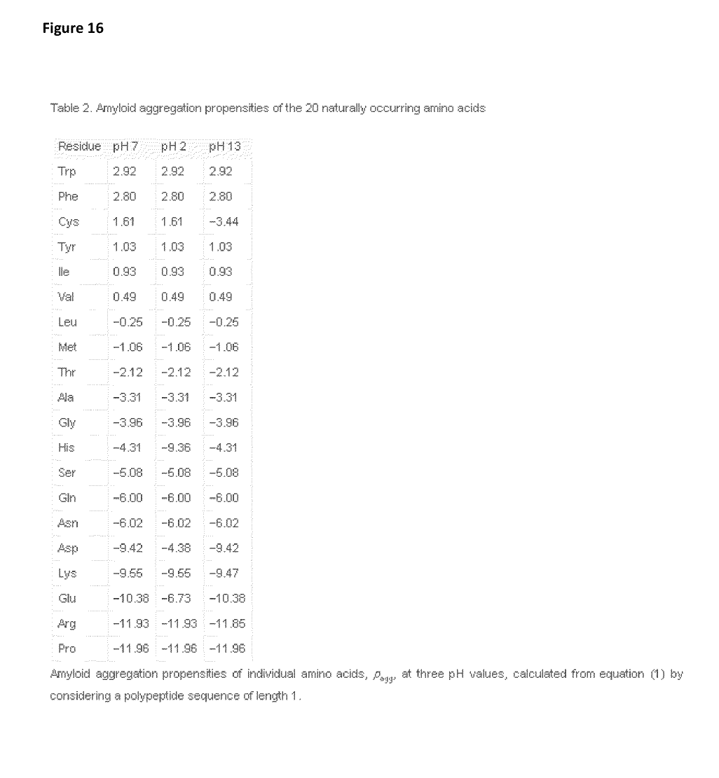

Analysis of material from the polyclonal pools showed that for the 15-8 molecule, both mutants were found to have improved solubility between pH 5-7 as compared to the parent (FIG. 14A). For 7r-29, the Y58N mutant molecules also exhibited improved solubility as compared to wild-type at this pH range; however mutation of the tryptophan residue at position 56 to an asparagine only improved solubility at pH5 and pH7 (FIG. 14B). Finally, both variants of the 21-23 molecule were found to have improved solubility at pH5, with the Y58N variant also exhibiting increased solubility at pH7 (FIG. 14C).

Within this specification the invention has been described, with reference to embodiments, in a way which enables a clear and concise specification to be written. It is intended and should be appreciated that embodiments may be variously combined or separated without parting from the invention.

### REFERENCES

Biswas, K. M., DeVido, D. R., and Dorsey, J. G. (2003) Evaluation of methods for measuring amino acid hydrophobicities and interactions. J. Chromatogr. A 1000:637-655.
Chennamsetty, N., Voynov, V., Kayser, V., Helk, B. and Trout, B. L. (2009) Design of therapeutic proteins with enhanced stability. Proc. Natl. Acad. Sci. USA 106:11937-11942.
Conchillo-Sole, O., de Groot, N. S., Aviles, F. X., Vendrell. J., Daura, X. and Ventura, S. (2007) AGGRESCAN: a server for the prediction and evaluation of “hot spots” of aggregation in polypeptides. BMC Bioinformatics 8:65
Eisenberg, D. (1984) Three-dimensional structure of membrane and surface proteins. Ann. Rev. Biochem. 53: 595-623.
Fernandez-Escamilla, A.-M., Rousseau, F., Schymkowitz, J. and Serrano, L. (2004) Prediction of sequence-dependent and mutational effects on the aggregation of peptides and proteins. Nature Biotechnol. 22:1302-1306.
Janin, J. (1979) Surface and inside volumes in globular proteins. Nature 277:491-492.
Kyte, J. and Doolittle, R. F. (1982) A simple method for displaying the hydropathic character of a protein. J. Mol. Biol. 157:105-132.
Maurer-Stroh, S., Debulpaep, M., Kuemmerer, N., de la Paz, M. L., Martins, I. C., Reumers, J., Morris, K. L., Copland, A., Serpell, L., Serrano, L., Schymkowitz, J. W. H. and Rousseau, F. (2010) Exploring the sequence determinants of amyloid structure using position-specific scoring matrices. Nature Methods 7:237-242.
Mendoza, R. P., Rubens, N., and Okamoto, T. (2010) Hierarchical Aggregation Prediction Method. KDD Cup 2010 Workshop, ACM SIGKDD Conference on Knowledge Discovery and Data Mining.
Pawar, A. P., Dubay, K. F., Zurdo, J., Chiti, F., Vendruscolo, M. and Dobson, C. M. (2005) Prediction of “aggregation-prone” and “aggregation-susceptible” regions in proteins associated with neurodegenerative diseases. J. Mol. Biol. 350:379-392.
Rose, G., Geselowitz, A., Lesser, G., Lee, R. and Zehfus, M. (1985) Hydrophobicity of amino acid residues in globular proteins. Science 229:834-838.
Rose, G. D. and Wolfenden, R. (1993) Hydrogen bonding, hydrophobicity, packing, and protein folding. Annu. Rev. Biomol. Struct. 22:381-415.
Trovato, A., Seno, F. and Tosatto, S. C. E. (2007) The PASTA server for protein aggregation prediction. Protein Engineering, Design & Selection 20:521-523
Wimley, W. C. and White, S. H. (1996) Experimentally determined hydrophobicity scale for proteins at membrane interfaces. Nature Struct. Biol. 3:842-848.
Wolfenden, R., Andersson, L., Cullis, P. M. and Southgate, C. C. B. (1981) Affinities of amino acid side chains for solvent water. Biochemistry 20:849.

1. A variant of a parent polypeptide comprising a V_{H} domain having a human germline framework, the variant comprising a V_{H} domain which differs from the V_{H} domain of the parent polypeptide by a substitution of at least one of amino acid positions 56, 57, 58, 59 and 79, wherein the amino acid at said at least one amino acid position of the variant polypeptide is more hydrophilic or has a reduced aggregation propensity than the substituted amino acid of the parent polypeptide.

2. The variant according to claim 1, wherein the at least one amino acid is more hydrophilic that the substituted amino acid.

3. The variant according to claim 1, wherein the at least one amino acid has a reduced aggregation propensity than the substituted amino acid.

4. The variant according to claim 1, wherein the variant has increased expression in a biological expression system when compared to the parent polypeptide.

5. The variant according to claim 1, wherein the substitution is of at least one of positions 56, 58 and 79.

6. The variant according to claim 1, wherein the substitution is of at least one of amino acid positions 56, 58 and 59.

7. The variant according to claim 1, wherein the substitution is of at least one, or both, of amino acid positions 56 and 58.

8. The variant according to Claim 1, wherein the substitution is at amino acid position 56.

9. The variant according to claim 1, wherein the amino acid at said at least one amino acid position of the variant polypeptide is an amino acid which exists in the corresponding position of a human V_{H} germline sequence.

10. The variant according to claim 9, wherein the human V_{H} germline sequences are selected from the VH3 subgroup.

11. The variant according to claim 9, wherein the parent polypeptide has the human DP-47 germline framework, and wherein said at least one amino acid is substituted with the amino acid which exists in the corresponding position of the human DP-2 germline sequence.

12. The variant according to claim 1, which comprises the amino acid sequence of amino acids 1-116 of SEQ ID NO:3 or SEQ ID NO:4.

13. A V_{H} domain comprising an amino acid sequence of amino acids 1-116 of SEQ ID NO:1 in which at least one of residues at positions Y56, T57, Y58, Y59 and Y79 of the V_{H} domain is substituted with a more hydrophilic residue, or with a residue having a lower propensity to aggregate.

14. An V_{H} domain according to claim 13, comprising an amino acid sequence of amino acids 1-116 of SEQ ID NO:3 or SEQ ID NO:4.

15. An antigen-binding construct comprising a protein scaffold which is linked to a variant polypeptide of claim 1, or a V_{H} domain of claim 13.

16. The antigen-binding construct of claim 15, wherein the protein scaffold comprises at least one domain of an human antibody constant region.

17. The antigen-binding construct of claim 16, wherein the protein scaffold comprises an Fc domain.

18. An isolated polypeptide having an amino acid sequence of SEQ ID NO:3 or SEQ ID NO:4.

19. A library comprising variant V_{H} domains according to claim 1 or antigen-binding constructs according to.

20-29. (canceled)
